(19) United States
(12) Patent Application Publication
(10) Pub. No.: US 2021/0319907 A1
(43) Pub. Date: Oct. 14, 2021

(54) MULTI-OMIC SEARCH ENGINE FOR INTEGRATIVE ANALYSIS OF CANCER GENOMIC AND CLINICAL DATA

(71) Applicant: HUMAN LONGEVITY, INC., SAN DIEGO, CA (US)

(72) Inventors: Alena HARLEY, SAN DIEGO, CA (US); Eve SHINBROT, SAN DIEGO, CA (US); Corine LAU, SAN DIEGO, CA (US)

(21) Appl. No.: 17/284,558

(22) PCT Filed: Oct. 14, 2019
(86) PCT No.: PCT/US2019/056166

§ 371 Date: Apr. 12, 2021

Related U.S. Application Data
(60) Provisional application No. 62/745,150, filed on Oct. 12, 2018.

(51) Int. Cl. G16H 50/70; G06F 16/22; G06F 16/2457; G06N 3/08; G16H 30/40; G16H 70/60

(52) U.S. Cl. CPC G16H 50/70; G06F 16/2272; G16H 70/60; G06N 3/08; G16H 30/40; G06F 16/24578

(57) ABSTRACT

A method is provided for utilizing multi-omic data indices for tumor profiling. The method can comprise storing a plurality of multi-omic data indices, wherein each of the plurality of multi-omic data indices comprises cancer-specific tokenized data; ingesting additional multi-omic data and any annotations associated with the additional multi-omic data, the additional multi-omic data related to one or more indices; indexing the ingested additional multi-omic data and annotations while preserving gene names, gene variant names and multi-omic mapping between different data streams for the same patient in the specific index, to produce tokenized ingested additional multi-omic data; receiving a user query; selecting one or more relevant multi-omic data indices based on the user query; ranking the selected one or more multi-omic data indices based on at least one of clinical actionability, pathogenicity, feature weight, or frequency; and returning the ranked one or more multi-omic data indices to the user.

### BACKGROUND

With the rising importance of cancer genomic sequencing, thousands of cancer genomes, exomes, transcriptomes, proteomes and other cancer data-omes are being sequenced by both private and public institutions (e.g. The Cancer Genome Atlas [TCGA], International Cancer Genome Consortium [ICGC]). The interpretation and analysis of the tumor and normal sequencing data is dependent on integrative analysis of both private and public genomic data and databases.

Industry, biopharmaceutical companies, research institutions and international cancer consortiums face hurdles such as, for example, (1) providing immediate access to any sample or a subset of samples; (2) integrating multi-omic data sets to form a complete picture of tumor biology; and (3) effectively associating prognostic, diagnostic and therapeutic information to all the available data (e.g., genomic, transcriptomic, proteomic, functional, medical, imaging, literature) to provide clinical insights and actionability for individual cancer patients as well as stratification of cohorts of patients on a potential multi-omic prognostic, diagnostic, or therapeutic biomarker(s).

Currently, publicly available data is scattered through publications, guidelines and web-based resources. Ultimately, the solution addressing problems such as the three outlined above will bring cancer genome analyses to widespread clinical use.

Data integration and harmonization poses a particularly acute challenge in cancer sequencing, namely, standardization and integration to allow the user to incorporate multiple sources of data and identify clinically and biologically relevant information. In addition, compared with germline sequence analysis, genomic analysis of cancer requires extensive bioinformatics pipelines and produces multi-omic streams of data for the same sample. For example, for a typical cancer biopsy and blood normal, binary base calls (BCL) for tumor DNA, normal DNA, tumor RNA, sometimes normal RNA has to be converted into variant calls format (VCF) via alignment to the reference genome, deduplication, re-alignment, and variant recalibration. Moreover, it is generally an industry standard to run multiple somatic variant callers to derive a consensus set of somatic single nucleotide variants (SNV) and small insertions and deletions (indels). Of further interest, for example, is copy number variant (CNV) detection for tumor, differential gene expression between tumor and normal RNA-Seq replicates, data processing to confirm that variants detected in somatic (tumor) DNA are also expressed in RNA, and pipelines that detect gene fusions. Of further interest is the use of tools that call large structural variants, as well as tools that perform advanced bioinformatics to annotate cancer alterations and compute relevant properties of the tumor (e.g. tumor mutation burden, genomic mutational signatures, microsatellite status, expressed neo-antigens, HLA-typing of normal genome) and to identify tumor alterations that are clinically relevant.

Modern cancer profiling technologies can easily generate 25 gigabytes of multi-omic data per sample, meaning that researchers conducting medium-sized cancer biomarker discovery studies are easily faced with terabytes of raw data. Identifying relevant biomarkers is thus akin to ‘finding a needle in a haystack’. Moreover, once an analysis pipeline is finished running, there is effectively no way to interact with the results to form new hypothesis.

The most common way to address currently the accessibility, multi-integration and actionability problems of cancer data is to design a portal to display pre-filtered data tables and analysis based on previously curated files and pre-computed workflows. Examples of portals include, Illumina BaseSpace Correlation Engine and Cohort Analyzer, WuXI nextCODE TCGA portal, cBioPortal, IntOGen, Tumorscape, Tumorportal, Xena, ICGC Data Portal, St. Jude PeCan, and Qiagen OmicSoft. These portals, however, generally restrict the types of questions that can be addressed and additional analyses that can be carried out. Moreover, the data is usually inaccessible for interrogation at many levels of the bioinformatics pipeline. Data in the portals is often pre-filtered, not integrated, and usually not ranked. In addition, most portals do not host individual user data. The few that allow users to upload their own data typically do not provide means to integrate the user's data with the portal data, or to derive advanced cancer analytics and make this data accessible and ranked in terms of clinical actionability, pathogenicity, feature weight, or frequency.

There is therefore a need to provide systems and methods that effectively and efficiently provide immediate access to any sample or a subset of samples. There also exists a need to provide systems and methods that effectively and efficiently integrate multi-omic data sets to form a complete picture of tumor biology. There further exists a need to provide systems and methods that effectively and efficiently associate prognostic, diagnostic and therapeutic information to all the available data (e.g., genomic, transcriptomic, proteomic, functional, medical, imaging, literature) to provide clinical insights and actionability for individual cancer patients and stratify cohorts of patients on potential multi-omic prognostic or therapeutic biomarker(s).

### SUMMARY

rofiling. The method can comprise storing a plurality of multi-omic data indices, wherein each of the plurality of multi-omic data indices comprises cancer-specific tokenized data. The method can further comprise ingesting additional multi-omic data any and annotations associated with the additional multi-omic data, the additional multi-omic data related to one or more indices. The method can further comprise indexing the ingested additional multi-omic data and annotations while preserving gene names, gene variant names and multi-omic mapping between different data streams for the same patient in the specific index, to produce tokenized ingested additional multi-omic data. The method can further comprise receiving a user query. The method can further comprise selecting one or more relevant multi-omic data indices based on the user query. The method can further comprise ranking the selected one or more multi-omic data indices based on at least one of clinical actionability, pathogenicity, feature weight, or frequency. The method can further comprise returning the ranked one or more multi-omic data indices to the user.

In accordance with various embodiments, a non-transitory computer-readable medium is provided in which a program is stored for causing a computer to perform a method for utilizing multi-omic data indices for tumor profiling. The method can comprise storing a plurality of multi-omic data indices, wherein each of the plurality of multi-omic data indices comprises cancer-specific tokenized data. The method can further comprise ingesting additional multi-omic data and annotations associated with the additional multi-omic data, the additional multi-omic data related to one or more indices. The method can further comprise indexing the ingested additional multi-omic data and annotations while preserving gene names, gene variant names and multi-omic mapping between different data streams for the same patient in the specific index, to produce tokenized ingested additional multi-omic data. The method can further comprise receiving a user query. The method can further comprise selecting one or more relevant multi-omic data indices based on the user query. The method can further comprise ranking the selected one or more multi-omic data indices based on at least one of clinical actionability, pathogenicity, feature weight, or frequency. The method can further comprise returning the ranked one or more multi-omic data indices to the user.

In accordance with various embodiments, a system is provided for utilizing multi-omic data indices for tumor profiling. The system can comprise an indexing unit. The indexing unit can comprise a storage element configured to store a plurality of multi-omic data indices, wherein each of the plurality of multi-omic data indices comprises cancer-specific tokenized data. The indexing unit can further comprise an indexing engine. The indexing unit can be configured to ingest additional multi-omic data and annotation associated with the additional multi-omic data, the additional multi-omic data related to one or more indices. The indexing unit can be further configured to index the ingested additional multi-omic data and annotation while preserving gene names, gene variant names and multi-omic mapping between different data streams for the same patient in the specific index, to produce tokenized ingested additional multi-omic data. The system can further comprise a user interface configured to receive a user query. The system can further comprise a query engine configured to select one or more relevant multi-omic data indices from the indexing unit based on the user query. The system can further comprise a ranking engine configured to receive the selected one or more relevant multi-omic data indices, and to rank the selected one or more multi-omic data indices based on at least one of clinical actionability, pathogenicity, feature weight, or frequency. The ranking engine can be further configured to return the ranked one or more multi-omic data indices to the user via the user interface.

In accordance with various embodiments, a system is provided for utilizing multi-omic data indices for tumor profiling. The system can comprise an indexing unit. The indexing unit can comprise a storage element configured to store a plurality of multi-omic data indices, wherein each of the plurality of multi-omic data indices comprises cancer-specific tokenized data. The indexing unit can further comprise an indexing engine. The indexing unit can be configured to ingest additional multi-omic data and annotation associated with the additional multi-omic data, the additional multi-omic data related to one or more indices. The indexing unit can be further configured to index the ingested additional multi-omic data and annotation while preserving gene names, gene variant names and multi-omic mapping between different data streams for the same patient in the specific index, to produce tokenized ingested additional multi-omic data. The system can further comprise a user interface configured to receive a user query. The system can further comprise a query engine configured to select one or more relevant multi-omic data indices from the indexing unit based on the user query. The query engine can be further configured to rank the selected one or more multi-omic data indices based on at least one of clinical actionability, pathogenicity, feature weight, or frequency. The query engine can be further configured to return the ranked one or more multi-omic data indices to the user via the user interface.

In accordance with various embodiments, a multi-omic cancer search engine system is provided for tumor profiling. The system can comprise a storage element configured to store a plurality of integrated multi-omic indices; an advanced cancer analytics software module; a mutli-omic indexing pipeline; a ranking engine reflecting clinical utility of multi-omic cancer alterations; a query engine that selects and combines relevant multi-omic indices and returns ranked multi-omic alterations for individual samples and cohorts of samples; and a user interface configured to receive a user query and perform a search on the cancer data.

Additional aspects will be evident from the detailed description that follows, as well as the claims appended hereto and the drawings.

### BRIEF DESCRIPTION OF THE DRAWINGS

The foregoing illustrative examples of various aspects and implementations provide an overview or framework for understanding the nature and character of the claimed aspects and implementations:

FIG. 1 illustrates an example of a system architecture for the multi-omic cancer search engine in accordance with various embodiments.

FIG. 2a illustrates an example of a multi-omic index organization, in accordance with various embodiments. FIG. 2b illustrates an example of a hierarchical propagation of annotations and ranking of variants, in accordance with various embodiments.

FIG. 3 illustrates an example of a set of cancer analytics pre-computed and calculated dynamically for individual samples and cohorts, in accordance with various embodiments.

FIG. 4a illustrates an example of a wide and deep model for learning variant ranking, in accordance with various embodiments. FIG. 4b illustrates an example of a Learning to Rank engine relying on a Deep Semantic Similarity Model (DSSM) for bio-medical data, in accordance with various embodiments.

FIGS. 5a and 5b together illustrate an example of a workflow for the operation of a query engine, in accordance with various embodiments.

FIG. 6 illustrates an example of a user interface in accordance with various embodiments. As illustrated, for example, a single search box allows users to enter different queries and receive ranked results.

FIG. 7 illustrates an example of search results obtained with a particular syntax, in accordance with various embodiments.

FIGS. 8a and 8b illustrate an example of search results obtained with a particular syntax, in accordance with various embodiments.

FIG. 9 illustrates an example of search results returned from a user query, in accordance with various embodiments.

FIG. 10 illustrates an example of search results returned from a user query, in accordance with various embodiments.

FIG. 11 illustrates an example of search results returned from a user query, in accordance with various embodiments.

FIG. 12 illustrates an example of search results returned from a user query, in accordance with various embodiments.

FIG. 13 illustrates is a block diagram of a computer system, in accordance with various embodiments.

FIG. 14 illustrates a flow chart of a method for utilizing multi-omic data indices for tumor profiling, in accordance with various embodiments.

FIG. 15 illustrates a system for utilizing multi-omic data indices for tumor profiling, in accordance with various embodiments.

FIG. 16 illustrates a system for utilizing multi-omic data indices for tumor profiling, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

This specification describe various exemplary embodiments of a multi-omic search engine for integrative analysis of cancer genomic and clinical data, and systems and methods associated therewith. The disclosure, however, is not limited to these exemplary embodiments and applications or to the manner in which the exemplary embodiments and applications operate or are described herein.

Unless otherwise defined, all technical terms used herein have the same meaning as commonly understood by one of ordinary skill in the art to which the embodiments disclosed herein belongs. As used in this specification and the appended claims, the singular forms “a,” “an,” and “the” include plural references unless the context clearly dictates otherwise. Any reference to “or” herein is intended to encompass “and/or” unless otherwise stated.

This disclosure describes systems and methods for operating a multi-omic search engine for integrative analysis of cancer genomic and clinical data, and can be referred to herein by the shorthand “Cancer Search” or “cancer search”.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well-known and commonly used in the art.

As used herein, “DNA” (deoxyribonucleic acid) refers to a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA.

It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc. A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ and NEXTSEQ Systems of Illumina and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

As used herein, the phrase “genomic features” can refer to a genome region with some annotated function (e.g., a gene, protein coding sequence, mRNA, tRNA, rRNA, repeat sequence, inverted repeat, miRNA, siRNA, etc.) or a genetic/genomic variant (e.g., single nucleotide polymorphism/variant, insertion/deletion sequence, copy number variation, inversion, etc.) which denotes a single or a grouping of genes (in DNA or RNA) that have undergone changes as referenced against a particular species or sub-populations within a particular species due to mutations, recombination/crossover or genetic drift.

As used herein, the term “biomarkers” refers to objectively measurable indicators of biological states.

As used herein, the term “pathogenicity” refers to a property of a genetic alteration that increases an individual's susceptibility or predisposition to a certain disease or disorder. Also referred to as a predisposing mutation, deleterious mutation, and disease-causing mutation.

As used herein, the term “germline” refers to tissue derived from reproductive cells (egg or sperm) that become incorporated into the DNA of every cell in the body of the offspring. A germline mutation may be passed from parent to offspring.

As used herein, the term “somatic” refers to genetic alteration acquired by a cell in the course of cell division. Somatic mutations differ from germ line mutations, which are inherited genetic alterations that occur in the germ cells.

As used herein, the term “codon” refers to a trinucleotide sequence of DNA or RNA that corresponds to a specific amino acid.

As used herein, the term “UI” is an acronym for user interface.

As used herein, the term “query time” refers to the point in time when user submits a query.

As used herein, the term “learning-to-rank” or “ranking engine” or “relevance-learning engine” refers to the application of machine learning, typically supervised, semi-supervised or reinforcement learning, in the construction of ranking models for information retrieval systems. Training data consists of lists of items with some partial order specified between items in each list. This order is typically induced by giving a numerical or ordinal score or a binary judgment (e.g. “relevant” or “not relevant”) for each item. The ranking model's purpose is to rank, i.e., produce a permutation of items in new, unseen lists in a way that is “similar” to rankings in the training data in some sense.

As used herein, the term “latent space” or “hidden space” refers to a space where the features lie.

As used herein, the term “embedding” refers to a mapping of a document (e.g., text, image, structured data) into a lower-dimensional latent space preserving objects main characteristics.

As used herein, the term “deep-and-wide model” refers to deep learning model that jointly trains a wide linear model (e.g., for memorization) alongside a deep neural network (e.g., for generalization).

As used herein, the term “language model” refers to a probability distribution over sequences of words.

As used herein, the term “transformer model” refers to deep learning models with the core idea self-attention—the ability to attend to different positions of the input sequence to compute a representation of that sequence.

As used herein, the term “BM25” refers to a broad family of statistical functions in information retrieval that consider the number of occurrences of each query term in a document or set of documents—i.e., term-frequency (TF)—and the corresponding inverse document(s), and ranks the set of documents based on the query terms appearing in each document, regardless of their proximity within the document.

As used herein, the term “RM3” refers to information retrieval model useful for both relevance and pseudo-relevance feedback.

As used herein, the term “DSSM” is an acronym that stands for Deep Semantic Similarity Model.

As used herein, the term “Siamese network” refers to an artificial neural network that uses the same weights while working in tandem on two different input vectors to compute comparable output vectors.

As used herein, the term “FDA” is an acronym for the U.S. Food and Drug Administration.

As used herein, the term “NCCN” is an acronym for the National Comprehensive Cancer Network.

As used herein, the term “COSMIC” is an acronym for the Catalogue of Somatic Mutations in Cancer.

As used herein, the term “TCGA” is an acronym for The Cancer Genome Atlas.

As used herein, the term “CPRA” is an acronym for chromosome, position, reference, and alternative.

As used herein, the term “SNV” is an acronym for single nucleotide variants.

As used herein, the term “CNV” is an acronym for copy number variants.

As used herein, the term “BCL” is an acronym for binary base call.

As used herein, the term “FASTQ” refers to a text-based format for storing both a biological sequence (usually nucleotide sequence) and its corresponding quality scores. Both the sequence letter and quality score are each encoded with a single ASCII character for brevity.

As used herein, the term “BAM” refers to a binary format for storing sequence data.

As used herein, the term “VCF” is an acronym that stands for variant call format and refers to the format of a text file used in bioinformatics for storing gene sequence variations.

As used herein, the term “EHR” is an acronym that stands for electronic health records.

As used herein, the term “ASCO” is an acronym that stands for the American Society of Clinical Oncology.

This disclosure describes various embodiments of a multi-omic search engine for integrative analysis of cancer genomic and clinical data, referred to herein by the shorthand “Cancer Search.” Cancer Search is an extension of work presented in U.S. patent application Ser. No. 15/465,454, entitled “Genomic Metabolic, and Microbiombic Search Engine,” filed on Mar. 21, 2017, the contents of which are herein incorporated by reference in their entirety.

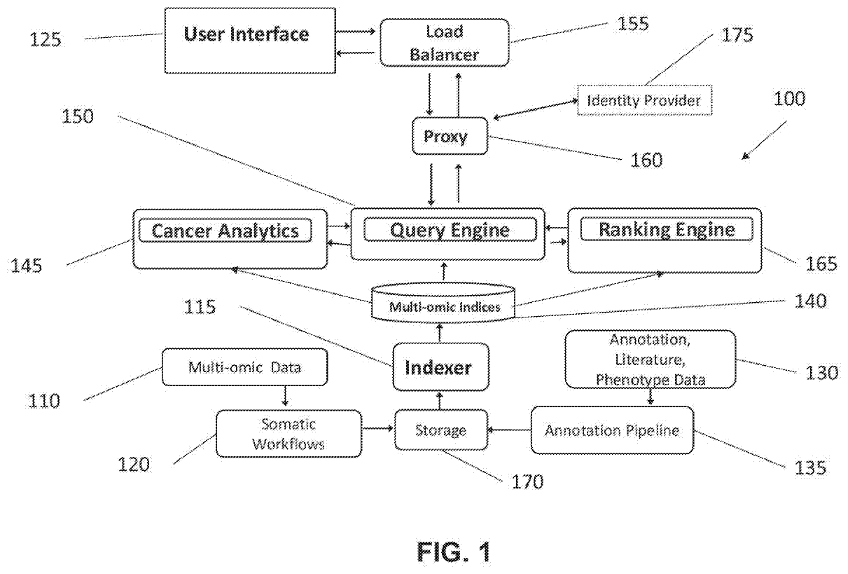

In accordance with various embodiments, a general search engine architecture is provided that can be configured to adapt to the specific needs for cancer multi-omic data. The general architecture, discussed below in more detail with reference to FIG. 1, can include various components. For example, the general architecture can include a web-based user interface, a query engine, an indexing pipeline that can index cancer multi-omic data with all annotations, a cancer analytics software module, and a ranking engine. The query engine can be configured to respond to requests to search any combination of multi-omic data streams available for individual samples or cohorts. The cancer analytics (e.g., in a software module or engine) can be configured to derive important tumor characteristics by pre-computing some characteristics and dynamically computing others at query time. The ranking engine can be configured such that, at indexing time, it will preload default clinically-actionable or pathogenicity-related ranking and, at query serving time, augment that ranking further based on detected query intent. More detail related to the various data types, pipelines, engines, modules, and analytics will be provided below.

The overall function of the user interface (UI) can be configured to present a unified and highly responsive way for querying and navigating the multi-omic cancer search results. The UI may actively maintain the state of the user search session. The UI can be configured to accept user queries, can relay them to the query engine, can render the resulting integrated multi-omic ranked results and their summary visualization if available, and can allow the user to interact with search results. The user can interact in various ways, via the UI, with the search results, including, for example, by providing relevance feedback e.g. promote/demote/pin/delete −type assessment of how well a result answers user information need, by comments on the accuracy of the information presented by a search result (e.g., a particular annotation source/publication being out of date or not being consistent), and by marking a particular result to be included in the dynamic individual patient or cohort report. More detail related to the UI will be provided below.

FIG. 1 represents a non-limiting example of a general architecture of a multi-omic cancer search system 100. A set of multi-omic data 110 (e.g., genomic, transcriptomic, etc.) for a sample(s) (e.g., tumor and/or normal sample) can be added to an indexing pipeline, or indexer 115, from a somatic workflow 120 or uploaded via a user interface 125. Non-limiting examples of upload formats can include FASTQs, BAMs, VCFs for tumor, normal, somatic VCF, RNA-Seq variant confirmation VCF, RNA-Seq differential gene expression in tabular format, CNV VCF, structural variants VCF, fusions calls VCF, or any combination thereof. The multi-omic data 110 can be cancer multi-omic data comprising BCLs, FASTQs, BAMs, VCFs, tabular cancer data, text cancer data, image cancer data. A set of annotation, literature and phenotypic data 130 can be added via annotation pipeline 135 to indexer 115. The data can either reside on a storage unit 170 (e.g., cloud storage, internal computer storage) or be uploaded by the user via specialized search upload interface. The data added by the indexing pipeline 115 can be stored in one or more indices 140. The system architecture can further include a cancer analytics engine or module 145 that can be configured to derive important characteristics of the tumor at indexing and serving time. Cancer analytics engine 145 can derive said important characteristics, regardless of whether the analysis is for individual samples or cohorts. The user interface 125 can allow a user to enter queries and receive results provided by a query engine 150. Query engine 150 can be configured to accept the user query; select, pre-join, aggregate, and summarize relevant multi-omic indices; and return ranked multi-omic data or features. In accordance with various embodiments, the system architecture can further include a load balancer 155 to accommodate the bi-directional transfer of data between UI 125 and query engine 150 for a large number of users. In accordance with various embodiments, the system architecture can further include an authenticating proxy 160, and include an identify provider 175 (e.g., a third party provider). The results retrieved from the indexer 115 can be ranked by a ranking engine 165 (e.g., learning-to-rank engine), which can be configured to derive a ranking model for, for example, variants, genes, pathways, phenotypes, text data, and images. The results retrieved from the indices can be ranked by the ranking engine and presented to the user in a ranked order. As will be discussed in detail herein, the data types that can be queried, analyzed, and ranked is vast, whether it be genomic, transcriptomic, epigenetic, chromatin accessibility data, microbiomic, proteomic, medical literature, phenotypic data, text data, imaging data, annotation sources, cancer analytics, prediction models, features contributing to the model accuracy, and so on. More detail will be presented below as to various method and system embodiments related to this example of a general architecture.

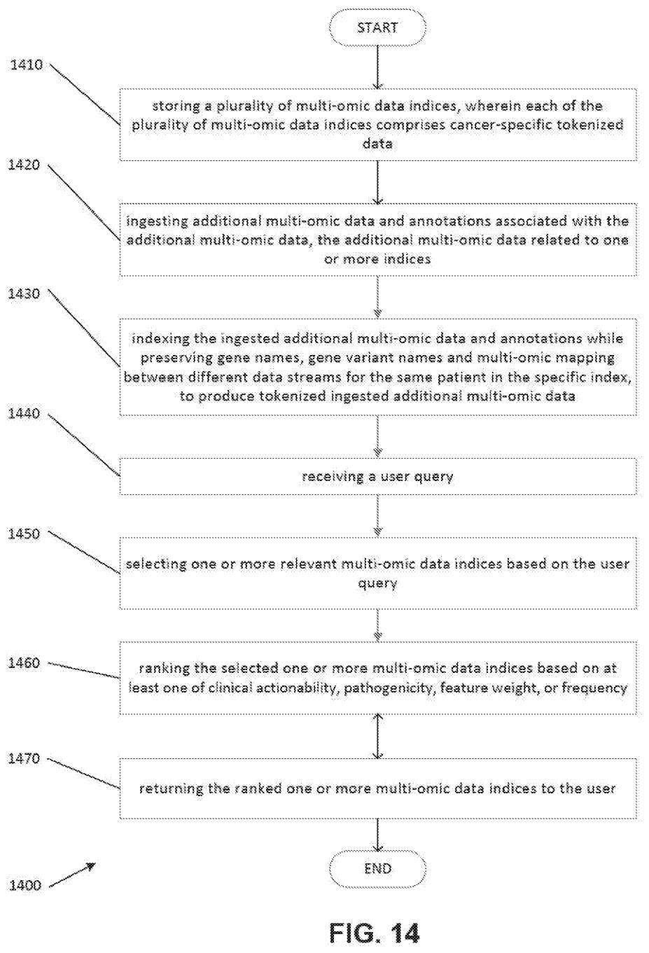

Referring now to FIG. 14, and in accordance with various embodiments, a method 1400 is provided for utilizing multi-omic data indices for tumor profiling. The method can comprise, at step 1410, storing a plurality of multi-omic data indices, wherein each of the plurality of multi-omic data indices comprises cancer-specific tokenized data. Further discussion related to, for example, storing features, multi-omic data indices, and cancer-specific data, are provided throughout this disclosure, and will be applicable to this and all embodiments discussed or contemplated herein.

The method can further comprise, at step 1420, ingesting additional multi-omic data and annotations associated with the additional multi-omic data, the additional multi-omic data related to one or more indices. Further discussion related to, for example, annotations and ingesting features are provided throughout this disclosure, and will be applicable to this and all embodiments discussed or contemplated herein.

The method can further comprise, at step 1430, indexing the ingested additional multi-omic data and annotations while preserving gene names, gene variant names and multi-omic mapping between different data streams for the same patient in the specific index, to produce tokenized ingested additional multi-omic data. Further discussion related to, for example, indexing, gene names, gene variant names and multi-omic mapping, are provided throughout this disclosure, and will be applicable to this and all embodiments discussed or contemplated herein.

The method can further comprise, at step 1440, receiving a user query. Further discussion related to, for example, receiving features and user queries, are provided throughout this disclosure, and will be applicable to this and all embodiments discussed or contemplated herein.

The method can further comprise, at step 1450, selecting one or more relevant multi-omic data indices based on the user query. Further discussion related to, for example, selection features, pre-joining of multi-omic indices, and relevancy determinations, are provided throughout this disclosure, and will be applicable to this and all embodiments discussed or contemplated herein.

The method can further comprise, at step 1460, ranking the selected one or more multi-omic data indices based on at least one of clinical actionability, pathogenicity, feature weight, and frequency. Other ranking factors such as, for example, factors related to query intent, can be included as well. Further discussion related to ranking is provided throughout this disclosure, and will be applicable to this and all embodiments discussed or contemplated herein.

The method can further comprise, at step 1470, returning the ranked one or more multi-omic data indices to the user. Further discussion related to, for example, returning features, displays and reports, are provided throughout this disclosure, and will be applicable to this and all embodiments discussed or contemplated herein.

In accordance with various embodiments, a non-transitory computer-readable medium in which a program is stored for causing a computer to perform a method for utilizing multi-omic data indices for tumor profiling. The steps within this method can be similar to that provided above, or can vary as needed.

The method can comprise storing a plurality of multi-omic data indices, wherein each of the plurality of multi-omic data indices comprises cancer-specific tokenized data. Further discussion related to, for example, storing features, multi-omic data indices, and cancer-specific data, is provided throughout this disclosure, and will be applicable to this and all embodiments discussed or contemplated herein.

The method can further comprise ingesting additional multi-omic data and annotations associated with the additional multi-omic data, the additional multi-omic data related to one or more indices. Further discussion related to, for example, annotations and ingesting features is provided throughout this disclosure, and will be applicable to this and all embodiments discussed or contemplated herein.

The method can further comprise indexing the ingested additional multi-omic data and annotations while preserving gene names, gene variant names and multi-omic mapping between different data streams for the same patient in the specific index, to produce tokenized ingested additional multi-omic data. Further discussion related to, for example, indexing, gene names, gene variant names and multi-omic mapping, is provided throughout this disclosure, and will be applicable to this and all embodiments discussed or contemplated herein.

The method can further comprise receiving a user query. Further discussion related to, for example, receiving features and user queries, is provided throughout this disclosure, and will be applicable to this and all embodiments discussed or contemplated herein.

The method can further comprise selecting one or more relevant multi-omic data indices based on the user query. Further discussion related to, for example, selection features and relevancy determinations, is provided throughout this disclosure, and will be applicable to this and all embodiments discussed or contemplated herein.

The method can further comprise ranking the selected one or more multi-omic data indices based on at least one of clinical actionability, pathogenicity, feature weight, or frequency. It should be noted that ranking can be further changed by the intent of a query (e.g., rank in order of reversed frequency, rank in order of feature contribution to the particular prediction of the model, rank mutational signature contributions in the reversed order of their weights, etc.). As such, clinical actionability can serve as a default ranking if other rankings are not requested and other intent is not easily (or cannot be) inferred. Further discussion related to, for example, ranking features and determinations, is provided throughout this disclosure, and will be applicable to this and all embodiments discussed or contemplated herein.

The method can further comprise returning the ranked one or more multi-omic data indices to the user. Further discussion related to, for example, returning features is provided throughout this disclosure, and will be applicable to this and all embodiments discussed or contemplated herein.

In accordance with various embodiments, the multi-omic data can be selected from the group consisting of genomic, transcriptomic, epigenetic, chromatin accessibility data, microbiomic, proteomic, phenotypic, image, relevant literature, integrated multi-omic data, and combinations thereof. In accordance with various embodiments, the plurality of multi-omic data indices can further comprise tumor (somatic) genomic alterations, normal (germline) genomic alterations, and cancer annotation sources.

In accordance with various embodiments, the methods discussed or contemplated herein can further comprise deriving cancer analytics for the selected one or more multi-omic data indices. The cancer analytics can comprise tumor characteristics selected from the group consisting of quality control, tumor mutation burden, genomic mutation signatures, microsatellite instability status, neo-antigens and their binding affinities, HLA-allele typing, RNA confirmed variants, copy number variants, structural variants, non-coding regulatory variants, gene fusions, pathway enrichment, cancer driver identification, mutation summary, differential gene expression, immune signatures, and combinations thereof. In accordance with various embodiments, the cancer analytics can be derived for an individual sample or a cohort of samples. Moreover, cancer analytics can include matching information about treatment outcomes for similar patients. In accordance with various embodiments, the cancer analytics can comprise machine learning predictions and ranked features. In accordance with various embodiments, the cancer analytics can comprise machine learning predictions and machine learning model features ranked in the order of their relevance to a particular prediction. The machine learning predictions can be selected from the group consisting of a primary site of origin classifier, a prediction of future metastasis site classifier, prediction of microsatellite instability status, prediction of neo-antigen binding affinities, disease state stratification, determining cancer lineages, and combinations thereof. The cancer analytics can be dynamically computed after receipt of the user query. The deriving of the cancer analytics can comprise utilizing deep neural networks and other machine learning methods (e.g. Support Vector Classifiers, Tree Methods, Ensemble Methods). The deriving of model feature importance can comprise gradient attribution methods or other feature importance methods

In accordance with various embodiments, the methods discussed or contemplated herein can further comprise propagating annotations from higher levels of genomic hierarchy to lower levels of genomic hierarchy.

In accordance with various embodiments, the methods discussed or contemplated herein can further comprise propagation of ranking for the selected one or more multi-omic data indices from higher levels of genomic hierarchy to lower levels of genomic hierarchy. The ranking can comprise a clinical ranking for cancer variants and genes. The ranking can comprise a probability of enrichment for genes belonging to a particular pathway. The ranking can comprise importance weight determined for features of the machine-learning model. The ranking can comprises stratifying a cohort by incorporating a latent space representation for cancer data and sub-selecting representations that result in the largest dis-entanglement between responders vs. non-responders, short- vs long-progression free survival, one vs another subtype of cancer, etc. The cohort can be stratified into responders and non-responders. The cohort can be stratified into long-progression free survival time and short-progression free survival time. The cohort can be stratified into different subtypes of cancer. The latent space representation can be performed by a neural network, or any other dimensionality reduction method (e.g. principal component analysis, individual component analysis, manifold learning). The neural network can be selected from the group consisting of autoencoders, variational autoencoders, deep belief networks, restricted Boltzman machines, feed forward, convolutional, recurrent, gated recurrent, long short-term memory, residual, and generative adversarial networks.

In accordance with various embodiments, including the methods discussed or contemplated herein, the ranking can further comprise a model for learning to rank selected from the group consisting of support vector machines, boosted decision trees, regression methods, neural networks, and combinations thereof. A model for learning to rank can also include other machine-learning models or deep neural networks. The ranking can further comprise deep learning ranking. The ranking can further comprise a similarity between embeddings of a query and indexed documents in a joint embedding space learned via deep learning methods. The deep learning ranking can be derived from a deep learning model selected from the group consisting of a deep semantic similarity model, a deep and wide model, a deep language model, a learned deep learning text embedding, a learned named entity recognition, Siamese neural network, and combinations thereof.

In accordance with various embodiments, including the methods discussed or contemplated herein, the multi-omic data can be selected from the group consisting of somatic (and germline) calls from whole genome sequence data, somatic (and germline) calls from whole exome sequence data, somatic (and germline) panel sequencing from fresh frozen tissue, somatic (and germline) panel sequencing from formalin-fixed paraffin-embedded tissue, somatic (and germline) panel sequencing from liquid biopsy, tumor and normal variant calls, tumor/normal transcriptomic data indexed as variant confirmed in RNA or gene expression level, epigenetic data, chromatin accessibility data, microbiomic data, proteomic data, single cell sequencing data, and combinations thereof. In various embodiments, the multi-omic data indexed can come from either an internal somatic calling and 16mmune pipeline, or be provided or uploaded in real time in the form of FASTQ, BAMs, VCFs and other tabular formats from any external partner.

In accordance with various embodiments, including the methods discussed or contemplated herein, the multi-omic data indices can further comprise extracted phenotypic data. The phenotypic data can be selected from the group consisting of electronic health records, clinical data, functional data, and combinations thereof.

In accordance with various embodiments, including the methods discussed or contemplated herein, the multi-omic data indices can further comprise featurized/embedded imaging data. The featurized imaging data can be selected from the group consisting of histology slides, MRI images, X-rays, mammograms, ultrasounds, PET images, CT scans, and combinations thereof.

In accordance with various embodiments, including the methods discussed or contemplated herein, the indexing of the ingested additional multi-omic data and annotation can further comprise indexing derived data selected from the group consisting of cancer analytics, annotations, features extracted from imaging data, phenotypic, medical literature data, data embeddings, and combinations thereof.

In accordance with various embodiments, including the methods discussed or contemplated herein, the ranking can further comprise matching sample alterations with established drug target labels and available clinical trials. The ranking can further comprise cancer drug target identification in cohorts by detecting a potential biomarker that stratifies the cohort based on a clinical variable of interest and/or statistical significance, and wherein returning the ranked one or more multi-omic data indices to the user comprises a stratification visualization.

In accordance with various embodiments, including the methods discussed or contemplated herein, the returning the ranked one or more multi-omic data indices to the user can further comprise a dynamic creation of hyper-linked reports (e.g., containing ranked alterations where each entry is hyperlinked to a search query) for individual patients and/or cohorts that provide comprehensive profiling of a tumor or cancer. Returning the ranked one or more multi-omic data indices to the user can further comprise returning a summary visualization of the returned results along with the list of ranked results.

In accordance with various embodiments, including the methods discussed or contemplated herein, the user query can comprise user-uploaded data selected from the group consisting of a panel of variants, genes, pathways, disease state conditions, phenotypes of interest, and wherein the selecting comprises querying individual sample or cohort data sub-selected by the uploaded data. The user query can be provided via a user interface, and can comprise uploading data for indexing selected from the group consisting of genomic data, transcriptomic data, epigenetic data, chromatin accessibility data, microbiomic data, proteomic data, phenotypic data, annotation data, and combinations thereof.

In accordance with various embodiments, the methods discussed or contemplated herein can further comprise normalizing and/or expanding the user query, classifying the intent of the query, summarizing retrieved documents, and performing document retrieval based on the similarity between the query and a document in a latent space using deep learning methods.

In accordance with various embodiments, including the methods discussed or contemplated herein, at least one of the indexing, selecting and ranking comprises utilizing deep neural networks.

In accordance with various embodiments, the methods (and systems) discussed or contemplated herein can operate to centralize a vast amount of cancer multi-omic data to provide a platform for oncologists, medical practitioners, research scientists, and other non-programmers to interrogate cancer bioinformatics pipelines at any level of detail and obtain clinical and biological insights into cancer biology and potential clinical treatment of cancer. Data types can include, for example, genomic (single nucleotide variations, indels in tumor and normal, structural rearrangements, copy number variations, gene fusions, and expressed variants for tumor genomes), transcriptomic, epigenetic, chromatin accessibility, microbiomic, proteomic abundance and localization, medical literature data (publications, treatment guidelines, clinical trials inclusion/exclusion criteria), phenotypic data (functional, clinical, electronic medical records, histopathology and radiology reports), imaging data (histopathology slides, MRI scans, X-rays, mammograms, ultrasounds, PET images, CT scans), cancer annotation sources (variants, genes, pathways, drugs), derived cancer analytics (tumor mutation burden, mutational signatures, microsatellite instability status, RNA sequence confirmed variants, differentially expressed genes, spatial omics lineage representations, neo-antigen binding affinities for MHC class I and class II molecule).

As discussed above and as will be discussed in further detail below, the various methods (and systems) described and contemplated herein, in accordance with various embodiments, include cancer analytics (e.g., as a step, feature, engine, module or software module). Cancer analytics allows users to access important characteristics of the tumor including, for example, tumor mutation burden, mutation signatures, spatial omics lineage representations, neo-antigen binding affinities for MHC class I and class II molecule, RNA sequence confirmed variants, differentially expressed genes, pathway enrichment, microsatellite instability status and microsatellite repetitive loci, and features extracted from imaging and clinical data. In accordance with various embodiments, this data can be pre-computed for individual samples or dynamically computed for cohort samples. In accordance with various embodiments, cancer analytics can provide for the integration of predictions from machine learning models and their features ranked by their contributions to a particular classification. Particular classifications can include for example, primary site of origin, prediction of future metastasis site, classifying variant as true or false positives, information about treatment outcomes for similar patients, outlier detection for sequencing quality, and disease state prediction for cohorts using latent and actual representations. The advantages of returning features ranked by their contributions to a particular classification, is that model predictions become more explainable to the user.

As discussed above and as will be discussed in further detail below, the various methods (and systems) described and contemplated herein, in accordance with various embodiments, include multi-modal ranking (e.g., as a step, feature, engine, module or software module). Multi-modal ranking can provide a relevance-learning engine to integrate multi-omic genetic data, annotation sources, literature data, clinical trial outcomes and significantly mutated genes in well characterized cohorts to learn clinically actionable ranking for cancer data. In various embodiments, machine learning models can be used to weigh contributions from annotations of multi-omic data. In various embodiments, deep learning and machine learning dimensionality reduction techniques can be used to derive latent space representation for cohorts of samples. In various embodiments, learned embeddings can be used for ranking genomic, text and imaging data.

As discussed above and as will be discussed in further detail below, the various methods (and systems) described and contemplated herein, in accordance with various embodiments, further include a mechanism (e.g., as a step, feature, engine, module or software module) for integrating and ranking multiple cancer annotation sources. These sources can include, for example, FDA labels, NCCN guidelines, clinical trials, CIViC, DoCM, OncoKB, Mycancergenome, Database of Genomic Biomarkers for Cancer Drugs, TCGA, ICGC, COSMIC, NCI60, CCLE, Drugbank, ClinVar, HGMD, PGMD, PharmGKB, dbSNP, dbNSFP, 1000Genomes, EXAC, CPDB, KEGG, BioCarta, BioCyc, Reactome, GenMAPP, MsigDB, Brenda, CTD, HPRD, GXD, BIND. In various embodiments, annotations and ranking can be propagated from a higher level of representation to the lower levels (e.g., for pathway to gene to variant, or from gene to variant codon to a full variant specification—chromosome, position, reference, alternative).

As discussed above and as will be discussed in further detail below, the various methods (and systems) described and contemplated herein, in accordance with various embodiments, further include a mechanism (e.g., as a step, feature, engine, module or software module) for integrating a number of deep learning models. The integration can function to provide neural data indexing (e.g., embedding multi-omic datasets, separately and together to regularize their respective latent space for DNA and RNA tumor alterations; embedding text data from electronic health records, clinical notes, literature, annotations; deep transformer models for named entity recognition and summarization of text and annotation data; embedding imaging data). The integration can further provide neural learning to rank models (e.g. Deep Semantic Similarity Model, Convolutional Deep Semantic Similarity Model, Recurrent Deep Semantic Similarity Model, Deep Relevance Matching Model, Interaction Siamese Networks, Lexical and Semantic matching networks, DeepRank) that can be used for addressing the feature engineering problem of learning-to-rank. The integration can provide neural querying models (e.g. deep learning transformer models for query normalization, synonym expansion, abbreviation expansion, term disambiguation, alternative suggestions. The integration can function to provide neural models for advanced cancer analytics (e.g., classification of a site of origin, prediction of the site of future metastasis, neoantigen binding affinities prediction, classifying variants as true or false positive, drug and trial matching, recommender systems for treatment that use information from similar cases indexed, models to compare decrease, increase, maintenance of allele fractions, copy number variation, RNA expression at each position for serial biopsies, and deep learning autoencoder methods and other dimensionality reduction techniques for cohort analytics and stratification).

As discussed above and as will be discussed in further detail below, the various methods (and systems) described and contemplated herein, in accordance with various embodiments, can further include (e.g., as a step, feature, engine, module or software module) statistical, machine learning and deep learning methods for identification of diagnostic, prognostic or predictive biomarker(s). When a user (e.g., academic or industry researcher) enters a phenotypic query on a cohort of samples, in various embodiments, ranked biomarkers are returned that can stratify a cohort, their statistical significance, and their summary visualization. In various embodiments, validation queries can be suggested by the search engine to perform robust algorithmic and statistical validation. In various embodiments, the system and methods can autosuggest iterative hypothesis refinement via suggested query refinement. In accordance with various embodiments, the statistical visualization and analysis derived for cancer cohort queries can include, for example, Kaplan-Meier survival analysis visualization, Log-rank test results visualization, Cox proportional hazards regression analysis visualizations, tree-structured survival models visualizations, heatmaps, scatter plots, box-plots and bar graphs providing statistical significance.

As discussed above and as will be discussed in further detail below, the various methods (and systems) described and contemplated herein, in accordance with various embodiments, can further include (e.g., as a step, feature, engine, module or software module) the use and/or receipt of an interactive summary visualization and/or ranked variants, genes, pathways, derived cancer analytics, outputs of integrated machine learning models (e.g., cancer type classification, most likely site of recurrence). This can be provided via a query engine (discussed in further detail below). In various embodiments, summary visualization can be dynamic and every data point can be linked to a particular result returned.

As discussed above and as will be discussed in further detail below, the various methods (and systems) described and contemplated herein, in accordance with various embodiments, can further provide interactive and fast access within 10000, 5000, 4000, 3000, 2000, 1000, 900, 800, 700, 500, 400, 300, 200, 100 milliseconds or less access, or any range of access in between the above values, to multi-omic cancer data ranked by clinical actionability, pathogenicity, feature weight, or frequency.

As discussed above, the systems and methods described herein, in accordance with various embodiments, can provide for a universal search interface (as opposed to many different entry points). In various embodiments, all knowledge, for example, multi-omic cancer data, samples, variants, genes, drugs, pathways, phenotypes, medical literature, image data, derived cancer analytics, machine learning models for predicting tumor characteristics and their features, upload of user's data, etc., can be accessible through the same simple search interface.

As discussed above and as will be discussed in further detail below, the various methods (and systems) described and contemplated herein, in accordance with various embodiments, can further provide (e.g., as a step, feature, engine, module or software module) the ability to compare sequential biopsy samples and provide difference (increase, decrease, maintenance) between old and new cancer drivers, variant allele fraction changes, copy number changes, and RNA confirmation status changes of cancer alterations.

As discussed above and as will be discussed in further detail below, the various methods (and systems) described and contemplated herein, in accordance with various embodiments, can further provide (e.g., as a step, feature, engine, module or software module) for various comparison regimes. These regimes can include, for example, (1) sample-to-sample comparison, comparison of any combination of multi-omic streams of data within the same patient, (2) sample to cohort comparison (e.g., compare individual sample to same cancer subtype in TCGA), and (3) pairwise cohort comparison (e.g., compare a cohort to a well characterized TCGA cohort with the same cancer type).

In accordance with various embodiments, the various methods (and systems) described and contemplated herein can provide (e.g., as a step, feature, engine, module or software module) for dynamic upload of a variant/gene drug target panel from the user's institution (or panels currently used in practice). Subsequent queries can indicate to use intersection of uploaded panel and multi-omic data stored for sample(s).

In the public domain, and as discussed already herein, a generic genomic search to address the problem of immediate access to germline genomic data has been proposed. It represents a significantly different problem of germline genome profiling that focuses on Mendelian rare variants, GWAS hits, burden tests and polygenic risks for common diseases, and inherited risks. To solve effectively all three main problems in comprehensive cancer characterization discussed above and herein, the systems and methods described herein, in accordance with various embodiments provided and contemplated, can further include the advanced cancer analytics for individual samples and cohorts, and a ranking engine (discussed in detail above and herein). The systems and methods described herein, in accordance with various embodiments provided herein, can augment all parts of an existing generic germline search system to integrate multi-omic data during indexing and serving time, to rank cancer alterations due to their clinical relevance and pathogenicity, and to make the search engine paradigm useful for comprehensive cancer profiling for individual samples and cohorts. In addition, the systems and methods described herein, in accordance with various embodiments provided herein, can include cancer cohort stratification analytics build on top of the cancer search engine that was absent from previous work in its entirety.

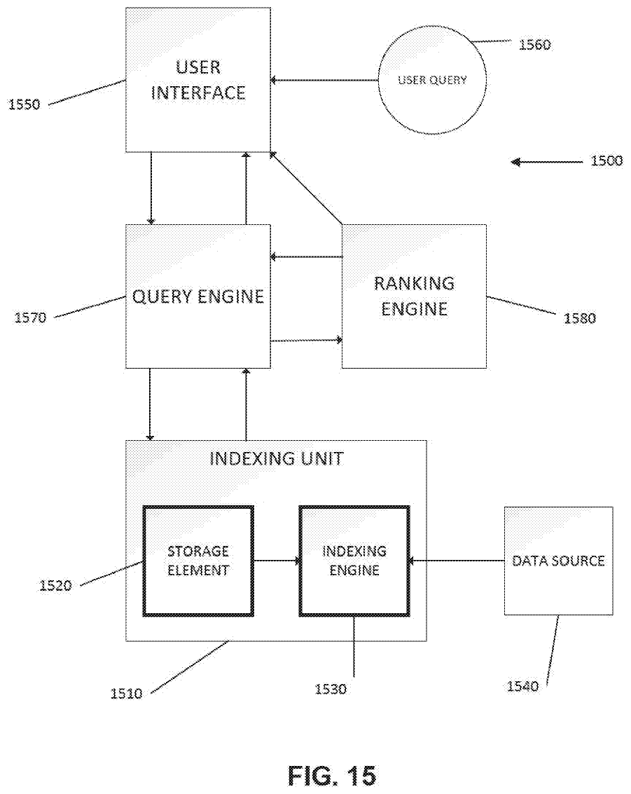

In accordance with various embodiments, FIG. 15 illustrates a system 1500 is provided for utilizing multi-omic data indices for tumor profiling. System 1500 can comprise an indexing unit 1510. The indexing unit can comprise a storage element 1520 configured to store a plurality of multi-omic data indices, wherein each of the plurality of multi-omic data indices comprises cancer-specific tokenized data. Indexing unit 1510 can further comprise an indexing engine 1530. Indexing unit 1510 can be configured to ingest additional multi-omic data and annotation associated with the additional multi-omic data via a data source 1540, the additional multi-omic data related to one or more indices. Indexing unit 1510 can be further configured to index the ingested additional multi-omic data and annotation from data source 1540 while preserving gene names, gene variant names and multi-omic mapping between different data streams for the same patient in the specific index, to produce tokenized ingested additional multi-omic data.

System 1500 can further comprise a user interface 1550 configured to receive a user query 1560.

System 1500 can further comprise a query engine 1570 configured to select one or more relevant multi-omic data indices from indexing unit 1510 based on user query 1560.

System 1500 can further comprise a ranking engine 1580 configured to receive the selected one or more relevant multi-omic data indices (e.g., from query engine 1570), to rank the selected one or more multi-omic data indices, and return the ranked one or more multi-omic data indices to the user via user interface 1550.

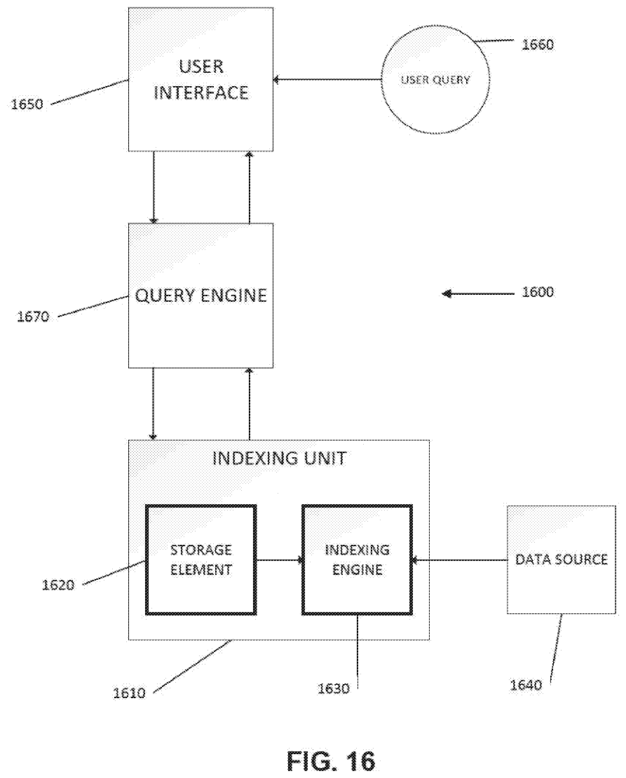

In accordance with various embodiments, FIG. 16 illustrates a system 1600 is provided for utilizing multi-omic data indices for tumor profiling. System 1600 can comprise an indexing unit 1610. The indexing unit can comprise a storage element 1620 configured to store a plurality of multi-omic data indices, wherein each of the plurality of multi-omic data indices comprises cancer-specific tokenized data. Indexing unit 1610 can further comprise an indexing engine 1630. Indexing unit 1610 can be configured to ingest additional multi-omic data and annotation associated with the additional multi-omic data via a data source 1640, the additional multi-omic data related to one or more indices. Indexing unit 1610 can be further configured to index the ingested additional multi-omic data and annotation from data source 1640 while preserving gene names, gene variant names and multi-omic mapping between different data streams for the same patient in the specific index, to produce tokenized ingested additional multi-omic data.

System 1600 can further comprise a user interface 1650 configured to receive a user query 1660.

System 1600 can further comprise a query engine 1670 configured to select one or more relevant multi-omic data indices from indexing unit 1610 based on user query 1660. Query engine 1670 can be further configured to rank the selected one or more multi-omic data indices based on clinical actionability, pathogenicity, feature weight, or frequency. The query engine can by further configured to return the ranked one or more multi-omic data indices to the user via the user interface 1650.

Note that all previous discussion of additional features, particularly with regard to the preceding described methods and non-transitory computer-readable media, in accordance with various embodiments, are applicable to the features of the various system embodiments described and contemplated herein.

In accordance with various embodiments, a computer-implemented system is provided for utilizing multi-omic data indices for tumor profiling. The system can comprise a computer storage, a digital processing device comprising at least one processor, an operating system configured to perform executable instructions, a memory, and a computer program including instructions executable by the digital processing device to create a multi-omic cancer search engine application. The multi-omic cancer search engine application can comprise a plurality of integrated multi-omic indices that are recorded in the computer storage and a software module providing advanced cancer analytics. The multi-omic cancer search engine application can comprise a software module providing multi-omic indexing pipeline ingesting multi-omic cancer data, annotation, medical and clinical data associated with the multi-omic genomic and imaging data, tokenizing the data while preserving variant nomenclature, gene names and drug names, and updating the indices with the tokenized data. The multi-omic cancer search engine application can further comprise a software module responsible for ranking integrated multi-omic data reflecting clinical utility of cancer alterations. The multi-omic cancer search engine application can comprise a query engine that selects and combines relevant multi-omic indices and returns ranked multi-omic alterations for individual samples and cohorts of samples. The multi-omic cancer search engine application can comprise a software module presenting a user interface allowing a user to enter a user query and perform faceted search on the multi-omic data.

In accordance with various embodiments, a non-transitory computer-readable storage media encoded with a computer program including instructions executable by a processor to create a multi-omic cancer search engine application is provided. The multi-omic cancer search engine application can comprise a plurality of integrated multi-omic indices that are recorded in the computer storage and a software module providing advanced cancer analytics. The multi-omic cancer search engine application can comprise a software module providing multi-omic indexing pipeline ingesting multi-omic cancer data, annotation, medical and clinical data associated with the multi-omic genomic and imaging data, tokenizing the data while preserving variant nomenclature, gene names and drug names, and updating the indices with the tokenized data. The multi-omic cancer search engine application can further comprise a software module responsible for ranking integrated multi-omic data reflecting clinical utility, pathogenicity, frequency, feature weight of returned results. The multi-omic cancer search engine application can comprise a query engine that selects and combines relevant multi-omic indices and returns ranked multi-omic alterations for individual samples and cohorts of samples. The multi-omic cancer search engine application can comprise a software module presenting a user interface allowing a user to enter a user query and perform faceted search on the multi-omic data.

In accordance with various embodiments, a computer-implemented method of providing a multi-omic cancer search engine application is provided. The multi-omic cancer search engine application can comprise a plurality of integrated multi-omic indices that are recorded in the computer storage and a software module providing advanced cancer analytics. The multi-omic cancer search engine application can comprise a software module providing a multi-omic indexing pipeline ingesting multi-omic cancer data, annotation, medical and clinical data associated with the multi-omic genomic and imaging data, tokenizing the data while preserving variant nomenclature, gene names and drug names, and updating the indices with the tokenized data. The multi-omic cancer search engine application can comprise a software module responsible for ranking integrated multi-omic data reflecting clinical utility of cancer alterations, pathogenicity, frequency, feature weight of returned results. The multi-omic cancer search engine application can comprise a query engine that selects and combines relevant multi-omic indices and returns ranked multi-omic alterations for individual samples and cohorts of samples. The multi-omic cancer search engine application can comprise a software module presenting a user interface allowing a user to enter a user query and perform faceted search on the multi-omic data. In various embodiments, the indices are optimally formatted in a partially pre joined configuration and clinical ranking is pre-loaded such that search speed is increased and a lag time between search and results is reduced. In various embodiments, pre-joining of multi-omic indices occurs before the user enters a query.

Note that all previous discussion of additional features, particularly with regard to the preceding described computer implemented methods, computer implemented systems, and non-transitory computer-readable media, in accordance with various embodiments, are applicable to the features of the various system embodiments described and contemplated herein.

As discussed above, the systems and methods described herein, in accordance with various embodiments, can centralize a vast amount of cancer multi-omic data comprising. That date can include, for example, genomic (e.g. single nucleotide variations, indels in tumor and normal; structural rearrangements, copy number variations, gene fusions, and expressed variants for tumor genomes), transcriptomic (e.g. RNA-Seq variant confirmation and differential gene expression), epigenetic, chromatin accessibility, microbiomic, proteomic abundance and localization, medical literature data (e.g. publications, treatment guidelines, clinical trials inclusion/exclusion criteria), phenotypic data (e.g. functional, clinical, EHR), imaging data (e.g. histology, MRI, X-rays, mammograms, ultrasounds, PET images, CT scans), cancer annotation sources (e.g. variants, genes, pathways, drugs), derived cancer analytics (e.g. tumor mutation burden, mutational signatures, microsatellite instability status, spatial omics lineage representations, neo-antigen binding affinities for MHC class I and class II molecule), predictions from machine learning models and their features (e.g. primary site of origin, microsatellite instability, site of potential future metastasis, drug and trial matches). In accordance with various embodiments, genomic data can be in the form of whole exomes, whole genomes, gene panel data, SNP arrays. In accordance with various embodiments, sequential biopsy multi-omic data may be indexed with the purpose of monitoring disease progression, development of drug resistance, and recurrence monitoring.

In accordance with various embodiments, the data indexed can be in the form of, for example and not limited to, Variant Call Format (VCFs), BAMs and FASTQs, for both the tumor and normal, or tumor only. In accordance with various embodiments, phenotypic data can be provided in a tabular format or in a raw format (e.g. EHR, clinical notes, pdf reports).

As discussed above, the systems and methods described herein, in accordance with various embodiments, can include annotation sources. Examples of annotation sources can include, but are not limited to: FDA labels, NCCN guidelines, clinical trials, CIViC, DoCM, OncoKB, Mycancergenome, Database of Genomic Biomarkers for Cancer Drugs, TCGA, ICGC, COSMIC, NCI60, CCLE, Drugbank, ClinVar, HGMD, PGMD, PharmGKB, dbSNP, dbNSFP, 1000Genomes, EXAC, CPDB, CADD, PolyPhen, dbNSFP, and many others.

The systems and methods described herein, in accordance with various embodiments, can also include drug target information, which can be derived and integrated from multiple sources. Those sources can include, for example and not limited to, FDA labels, NCCN Drug and Biologics Compendium, Thomson Micromedex DrugDex, Elsevier Gold Standard's Clinical Pharmacology compendium, American Hospital Formulary Serving-Drug Information Compendium, ESMO guidelines, ASCO guidelines, NCCN guidelines, and mutations annotated in other cancer knowledge databases such as, for example, OncoKB, CIViC, DoCM, COSMIC. In accordance with various embodiments, drug targets can be indexed on variant, gene, and pathway levels. In accordance with various embodiments, drug indication, evidence, cancer type, adverse reactions reported and additional information can be stored in search indices.

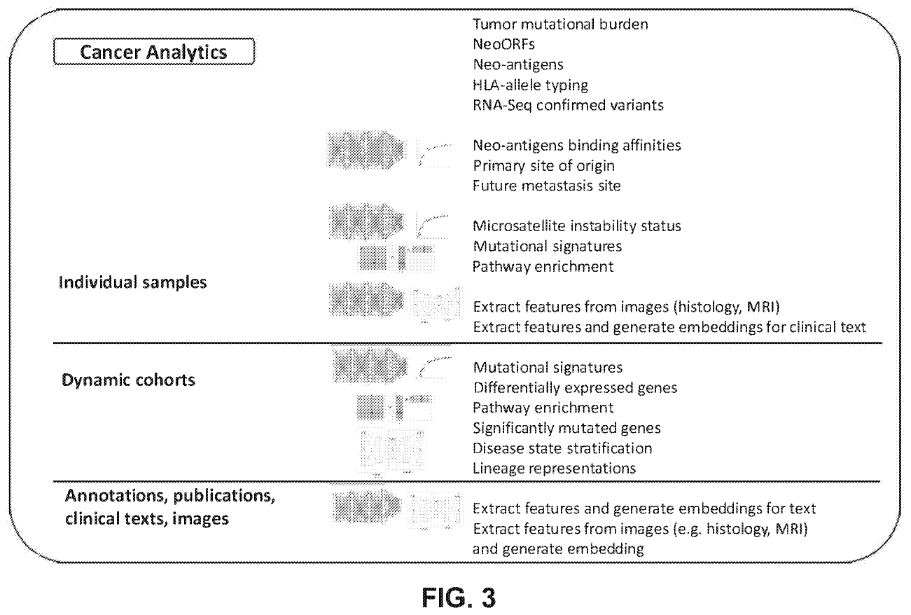

As discussed above, the systems and methods described herein, in accordance with various embodiments, can include cancer analytics (or advanced cancer analytics), or a software module providing advanced cancer analytics, or use of the same. The software module can provide derived cancer analytics both pre-computed (e.g., computed at the indexing time) and dynamic (e.g., computed at the query time). In accordance with various embodiments, the advanced analytics can also be visualized at the query time. FIG. 3 illustrates an example of cancer analytics pre-computed and calculated dynamically for individual samples and cohorts. An advanced analytics module can integrate predictions from machine learning and deep learning models for predicting important characteristics of tumor biology.

In accordance with various embodiments, precomputed derived cancer analytics for individual samples can include, for example and not limited to, tumor mutational burden (an important biomarker for therapy, e.g., immune therapy), microsatellite instability status (an important cancer state where mismatch repair proteins are disabled), genomic mutational signatures (potentially etiological and mechanistic bases for cancer), detected neoORFs (frameshift mutations that may lead to novel amino acid sequences that can be useful for cancer vaccines), detected neo-antigens, neo-antigen binding affinities for MHC class I and class II molecule, HLA allele typing (an important variable for cancer vaccine design), expressed immune genes (e.g., genes that play role in response to the immunotherapy treatments), RNA sequence confirmed variants, and differentially expressed genes.

In accordance with various embodiments, dynamic advanced cancer analytics for individual samples can include, for example and not limited to, pathway enrichment analysis for a specific type of variants (based on a query, e.g. non-silent variants), and spatial omics lineage representations. In accordance with various embodiments, dynamic advanced cancer analytics for a cohort of samples can include, but is not limited to, cohort mutational signatures; detection of significantly mutated genes and cancer drivers by collapsing recurrent somatic alterations in the same gene and after correcting for ratio of non-silent to silent variants, gene replication time, and other properties of cancer biology; disease state stratification; spatial omics lineage representations; and pathway enrichment analysis for a subset of variants (e.g. non-silent mutations).

In accordance with various embodiments, cancer analytics can be provided via an advanced analytics module that can be configured to integrate predictions from, for example, machine learning and deep learning models for predicting important characteristics of tumor biology (e.g., tumor only and tumor-normal classifier for microsatellite instability status; tumor origin classification for metastatic tumors of unknown origin; models for predicting most likely recurrence site for a particular patient; deep learning and machine learning methods for tumor only variant calling; neoantigen binding prediction; machine learning models for inherited cancer risk predictions for different cancer types; machine learning models for immunotherapy outcome predictions; classifying variants as true positive or false positives; deep learning methods for variant, gene, drug, and disease; named entity recognition for processing literature, EHR and clinical trial data; deep learning methods for identifying region of interest and extracting features from unstructured histology and radiology slides and other imaging data; deep learning models for learning latent embedding of cancer multi-omic disease states; deep learning methods for drug and trial matching; machine learning models for identifying similar patients; recommender systems for cancer treatments based on outcomes from treating similar patients; and machine learning and deep learning methods for cohort biomarker(s) stratification and cohort disease state identification).

The systems and methods described herein, in accordance with various embodiments, can include, for example, deep learning embeddings of phenotypic data (e.g., learned from electronic health records, clinical and functional records), annotation sources, medical literature or imaging data (e.g. histology slides, MRI, X-rays, mammograms, ultrasounds, PET images, CT scans).

The systems and methods described herein, in accordance with various embodiments, can include an advanced cancer analytics module setting statistical thresholds on quality control, identifying outliers for indexed sequencing quality metrics. Some non-limiting examples of quality control metrics of interest can include quality control for tumor-normal match (e.g., kinship and identity values); tumor and normal sequencing metrics (e.g., Freemix/Conpair metrics reflecting potential tumor/normal contamination, sequencing metrics that include, but are not limited to, mean total coverage, percent reads aligned, percent duplication, and Y/X ratio; and somatic sequencing quality control metrics that includes, but is not limited to, number of variants in dbSNP, dbSNP enrichment, dbSNP insertion deletion ratio, dbSNP transition/transversion ratio, and heterogeneous/homogeneous variant ration (heterozygous/homozygous variant ratio).

In accordance with various embodiments, the advanced cancer analytics (or its associated module) can provide, for example, dynamic algorithms for mutation summary, cancer driver identification, comparison of multiple biopsies, and cohort stratification based on the suspected (multi-omic) biomarker in cohorts of samples. In various embodiments, a comparison of a sample versus a cohort of samples can be implemented, as well as comparison of multiple cohorts.

The systems and methods described herein, in accordance with various embodiments, can include the indexing and centralizing of a vast amount of cancer multi-omic data. As discussed above in some detail, the data can include for example, and not limited to, genomic data (e.g., single nucleotide variations, indels in tumor and normal, structural rearrangements, copy number variations, gene fusions, and expressed variants for tumor genomes), transcriptomic data, epigenetic data, chromatin accessibility data, microbiomic data, proteomic abundance and localization data, medical literature data (e.g., publications, treatment guidelines, clinical trials inclusion/exclusion criteria), phenotypic data (e.g., functional, clinical, EHR), imaging data (e.g., histology slides, MRI, X-rays, mammograms, ultrasounds, PET images, CT scans), cancer annotation sources (e.g., variants, genes, pathways, drugs), derived cancer analytics (e.g., tumor mutation burden, mutational signatures, differentially expressed genes, spatial omics lineage representations, predictions and features from machine learning models of primary origin site, site of future metastasis, microsatellite instability status, neo-antigen binding affinities for MHC class I and class II molecule).

Applicants have advantageously found that by indexing raw data along with derived analytics, predictions from machine learning and deep learning models and their (derived) features and embeddings may include better machine learning interpretability, iterative hypothesis generation and refinement of successive queries by the user to characterize and understand tumor biology better.

In accordance with various embodiments, and as discussed above, the systems and methods disclosed herein can include a software module for mutli-omic indexing of cancer data, annotation, medical and clinical data associated with the genomic and imaging data, tokenizing the data while preserving variant nomenclature, gene names and drug names, and updating the indices with the tokenized data. In accordance with various embodiments, the step of mutli-omic indexing can include the integration and pre-joining of multi-omic indices on a level of a variant, gene, pathway, cancer subtype or sample.

Specific to cancer annotation data, the systems and methods described herein, in accordance with various embodiments, can include an indexing step (see above), or a software module providing mutli-omic indexing for cancer annotation data. Cancer annotation data can include, but is not limited to, FDA labels and NCCN guidelines, clinical trials, public cancer databases (CIViC, DoCM, OncoKB, Mycancergenome, COSMIC, Database of Genomic Biomarkers for Cancer Drugs, ICGC, TCGA), pubic genomic databases (ClinVar, dbNSFP, dbSNP), commercial data sources (HGMD, PGMD, PharmGKB, CPDB). In another aspect, multiomix-indexing software module indices also non-cancer focused annotation sources: ClinVar, dbNSFP, dbSNP, CPDB, HGMD, PGMD. In accordance with various embodiments, the software module for mutli-omic indexing can be configured to integrate and pre-join multi-omic annotation data on a level of a variant, gene codon number, gene, pathway, cancer subtype or sample.

In accordance with various embodiments, indexing can further include utilizing derived content embeddings to index complex phenotypes, literature data, histopathology, MRI, X-rays, mammograms, ultrasounds, PET images, CT scan images.

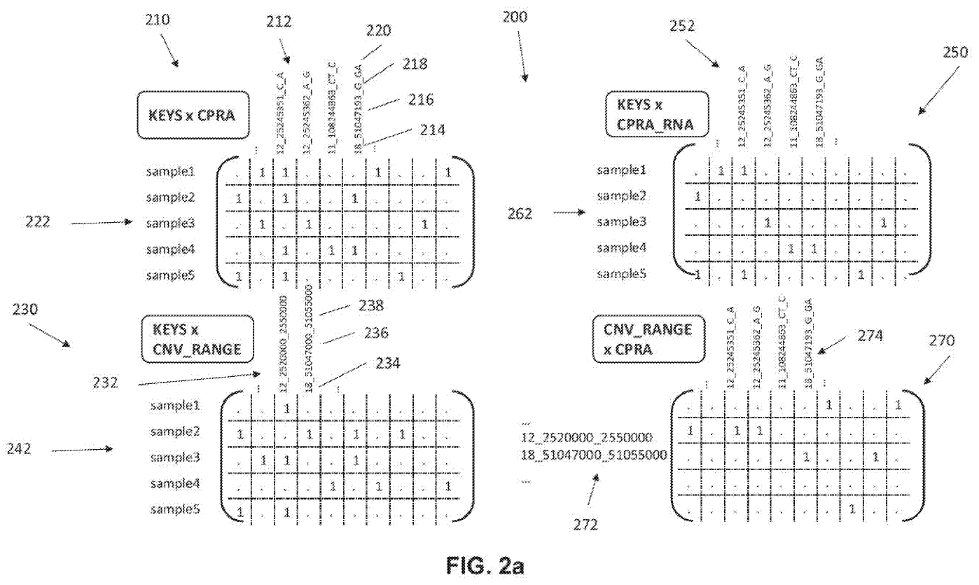

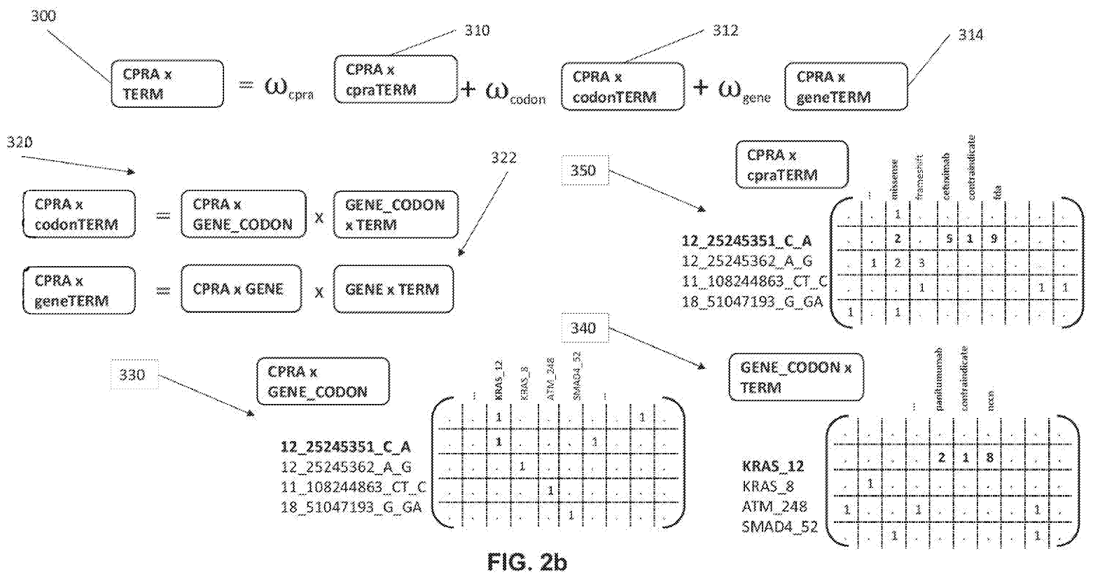

The systems and methods described herein, in accordance with various embodiments, can further include indexing procedures where multi-omic data integration during indexing takes place first at the sample level, and then at either the variant, gene codon number, gene or pathway level or any combination thereof as depicted in FIGS. 2a and 2b. In the non-limiting example of a multi-omic indexing integration illustrated in FIG. 2a, ingested multi-omic cancer data is selected from the group consisting of single nucleotide variants (SNVs) and small indels (represented as chromosome number, chromosomal position, reference, alternate allele—CPRA), copy number variants (CNV), and variants confirmed in RNA. SNVs can be indexed from somatic VCF-containing SNVs and small indels. Copy number variants (CNVs) called on a chromosomal regions (e.g., also mapped on a gene level using advanced cancer analytics module) can be indexed from copy number calls VCF (CNVs are also mapped on a gene level). RNA-Seq confirmed variants can be obtained from RNA-Seq analysis (derived from advanced cancer analytics module). Multi-omic indices can be joined to answer complex queries (e.g., get SNVs and small indels overlapping CNV gains and losses, expressed in RNA for a group of samples). Differentially expressed genes can be derived, for example, from an advanced analytics software module.

In accordance with various embodiments, joined multi-omic indices can be produced via selected indexing method such as, for example and not limited to, KEYSxCPRA, KEYSxCNV, KEYSxCNV_RANGE, KEYSxCNV_GENE, KEYSxCPRA_RNA, and KEYSxGENE_RNA for indexing of Copy Number Variants and confirmed RNA variants and (see again, FIG. 2a). Applicant has advantageously found that cross-indexing of multiple streams of information provided the ability to, for example, query any combination of multi-omic streams of data or the individual streams themselves, and perform entity linking at variant, gene codon number, gene, pathway and other levels.

Referring to the illustrated example on FIG. 2a, a first index table 210 describes single nucleotide polymorphisms and small indels in DNA in terms of their CPRA 212 (chromosome 214, position 216, reference 218, alternative allele 220) occurring in samples with KEYS sample IDs 222. A second index table 230 describes copy number variants (CNV) in terms of their ranges 232 (chromosome 234, beginning 236, end 238) occurring in samples with KEYS sample IDs 242. A third index table 250 describes variants in DNA (CPRA) 252 (see first index table 210) in terms of RNA-Seq occurring in samples with KEYS sample IDs 262. A fourth index table 270 describes copy number variants CNV 272 with their ranges versus single nucleotide polymorphisms and small indels in DNA (CPRA) 274.

Referring to illustrated example on FIG. 2b, a CPRAxTERM ranking 300 is provided and is composed of ranking for annotations (terms) aggregated on CPRA level 310, GENE_CODON level 312, and GENE level 314. Formula 320 provides an example for how to compute rank on GENE_CODON level for CPRA. Formula 322 provides an example for how to compute rank on GENE level for CPRA. A fifth index table 330 provides an example of a CPRA by GENE_CODON mapping index table. A sixth index table 340 provides an example of a GENE_CODON level annotations index table. A seventh index table 350 provides an example of a CPRA level annotations index table.

As discussed above, the systems and methods described herein, in accordance with various embodiments, can provide for the ranking of selected one or more multi-omic data indices. In various embodiments, ranking can occur without an associated filtering of the available cancer multi-omic data. Accessible data, as discussed above, can include, for example, variants, genes, pathways, RNA sequence confirmed variants, differentially expressed genes, hyper/hypo methylated regions, expressed proteins, copy number variants, structural variants, gene fusions, phenotypes, family history, annotations, drugs, clinical trial inclusion/exclusion criteria, derived analytics (e.g., mutational signatures weights, microsatellite repetitive loci, features extracted from imaging data and images themselves, and literature data and its embeddings), and machine learning models predictions and their features (e.g., microsatellite instability status and microsatellite instable loci, predicted primary site of origin and alterations identified as key-features of this model in order of their relative importance, predicted site of metastasis and key features of the model, and predicted neo-antigen binding affinities for MHC class I and class II molecules). In various embodiments, any combination of the different multi-omics streams or individual data streams can be returned based on the user query.

FIG. 2b, for example, illustrates an example of a hierarchical propagation of annotations and ranking for variants (CPRA) accumulated by weighted ranking of variant-level CPRA×cpraTERM, codon-level CPRA×codonTERM, and gene level CPRA×geneTERM annotations.

As discussed above, the systems and methods described herein, in accordance with various embodiments, can provide for the integrating and ranking of multiple cancer annotation sources. These multiple cancer annotation sources can include, for example, FDA labels, NCCN guidelines, NCCN Compendium biomarkers, clinical trials, CIViC, DoCM, OncoKB, Mycancergenome, Database of Genomic Biomarkers for Cancer Drugs, TCGA, ICGC, COSMIC, NCI60, CCLE, DrugBank, ClinVar, HGMD, PGMD, PharmGKB, dbSNP, dbNSFP, 1000Genomes, EXAC, CPDB, KEGG, BioCarta, BioCyc, Reactome, GenMAPP, MSigDB, Brenda, CTD, HPRD, GXD, and BIND.

In accordance with various embodiments, a multi-modal ranking engine (or module) can further include a relevance-learning engine to integrate, for example, annotation sources, literature data, clinical trial outcomes and significantly mutated genes in well characterized cohorts (such as TCGA) to learn clinically actionable ranking for multi-omic data in both individual patient and cohort query use case setting. In other embodiments, ranking learned can be based on predicted pathogenicity of alterations with unknown clinical significance.

As discussed above, the systems and methods described herein, in accordance with various embodiments, can provide for the ranking of cancer genomic alterations in terms of their clinical actionability, pathogenicity, feature weight, or frequency. In accordance with various embodiments, the ranking model can be derived by training a supervised learning model by learning to weigh features extracted for multi-omic cancer data. For variants (e.g., at exact position and a specific codon) or genes (e.g., mutation types are taken into account), this can include, for example, an indicator whether a variant/or type of alteration in a gene has been implicated in FDA labels, NCCN guidelines, NCCN biomarker compendium, ASCO guidelines, ESMO guidelines or other top-tier cancer guidelines, and whether there is indication/contra-indication of a specific drug; features extracted for a variant/or type of alteration in a gene from other cancer annotation sources such as, for example, clinical trials, OncoKB, Mycancergenome, CIViC, DoCM, and Database of Genomic Biomarkers for Cancer Drugs; features extracted from other relevant annotation sources such as, for example, TCGA, TCGA significantly mutated genes, COSMIC cancer gene census, COSMIC, ICGC, Drugbank, Swissprot, dbNSFP, HGMD, PGMD, PharmGKB, and ClinVar; population allele frequency data from HLI, HLI cancer, TCGA, COSMIC, ICGC, 1000Genomes, EXAS, Gnomad; embeddings from text extracted from relevant clinical trials, PubMed, Medline, OMIM articles and other medical literature; and embeddings for the named entities extracted from the medical texts.

In accordance with various embodiments, ranking can be based on the Support Vector Regression, Boosted Trees, other machine learning model that weights information from annotation sources such as, for example, FDA, NCCN guidelines, NCCN biomarker compendium, curated cancer genes, COSMIC, TCGA Significantly Mutated Genes, known hotspots, clinical trials, and in silico predicted loss/gain of function scores (e.g. CADD, FATHMM, SIFT, Polyphen).

In accordance with various embodiments, three learning-to-rank methods are used to derive ranking. These methods include pointwise (e.g., logistic regression), pairwise (e.g., RankSVM, RankBoost) and listwise approaches (LambdaMart).

In accordance with various embodiments, ranking for variants and genes can be learned separately than ranking for other documents (e.g., medical literature), where a separate learning-to-rank model is trained to use weighted transformed feature sets that can include, for example, BM25, PageRank, RM3, and other ranking models for text documents.

In accordance with various embodiments, ranking for variants and genes can be learned separately, or as part of deep-and-wide modes together with ranking for other document types. In some embodiments, ranking for text documents utilizes deep learning language modelling (LM) ranks items by probability of document given a query. In accordance with various embodiments, the deep learning language model can be a transformer model (e.g., BERT, RoBERTa, Xlnet, Albert) fine-tuned on relevant data. Such models can be large scale, pre-trained language model embeddings. In accordance with various embodiments, document relevance can be generated using textual and temporal parts of documents, for example, by deriving multiple classes of features including, for example, entity features and time features both derived from a set of annotations, named entity recognition (NER), and temporal tagging.

In accordance with various embodiments, to provide additional semantic understanding, the deep learning methods (e.g., Deep Semantic Similarity Model, Convolutional Deep Semantic Similarity Model, Recurrent Deep Semantic Similarity Model, Deep Relevance Matching Model, Interaction Siamese Networks, Lexical and Semantic matching networks, Long short-term Memory networks, Transformer networks, Word embedding methods, DeepRank) can be used for addressing the feature engineering task of learning-to-rank, by primarily using automatically learned features from raw text of the query and the document. As such, deep learning methods can use neural networks of different types, whether it be, for example, convolutional or recurrent.

As discussed above, in accordance with various embodiments, the ranking can include a clinical ranking for cancer variants and genes. The ranking can include a deep learning ranking, wherein the deep learning ranking can be derived from a deep learning model selected from the group consisting of a deep semantic similarity model, a deep-and-wide model, a deep language model, a learned deep learning text embedding, a learned named entity recognition, Siamese neural network, and combinations thereof.

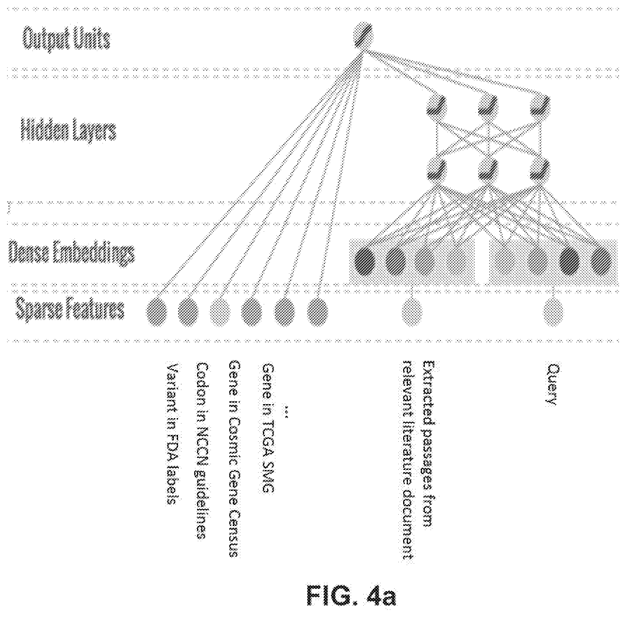

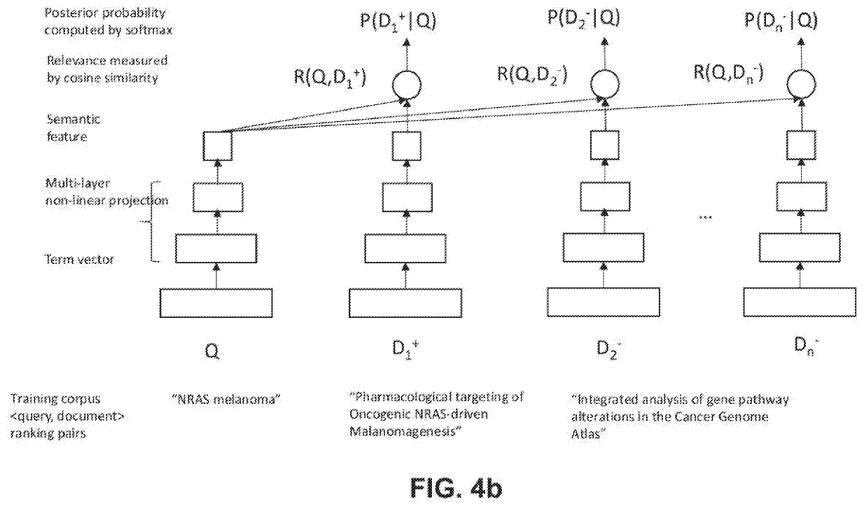

FIG. 4a illustrates an example of a wide and deep model for learning variant ranking. The wide part can effectively memorize sparse features and their interactions using cross-product feature transformations from different annotation sources, while the deep part can generalize to previously unseen feature interactions and literature embeddings.

FIG. 4b illustrates an example of a learning-to-rank engine relying on Deep Semantic Similarity Model (see above discussion) for bio-medical data. In the particular example illustrated in FIG. 4, a Siamese network is used to allow to learn semantic similarity between query (Q) and relevant documents (D⁺) by learning a joint query and document embedding. Relevance can be estimated by cosine similarity between query and document embeddings R(Q,D). The network can minimize cross entropy loss against randomly sampled negative documents

$$ℒ ( q , {d}^{+} , {D}^{-} ) = - log ( \frac{{e}^{γ \cdot cos ( \overline{q} , {\overline{d}}^{+} )}}{{Σ}_{d ∈ D} {e}^{γ \cdot cos ( \overline{q} , \overline{d} )}} ) where , D = \{ {d}^{+} \} ⋃ {D}^{-} .$$

After the ranking model is trained, document embedding can be pre-computed (e.g., as a centroid of all unit vectors of the words in the document). At query time, query vector embedding can be generated before assessing the similarity between query and document representations in a joint latent space. Note that specific queries and documents referenced in FIG. 4b are exemplary only and in no way limiting to the types of queries submitted and documents analyzed.

In accordance with various embodiments, global ranking can be optimized for clinical actionability (or pathogenicity when clinical utility is unknown) and preloaded into the indices, whereby results (subjected to, for example, a top-K algorithm) can be re-ranked to further satisfy a particular information need. In accordance with various embodiments, re-ranking can involve the use of language modeling or weighted transformed features from standard information retrieval models (e.g. PageRank, BM25, RM3).

In accordance with various embodiments, ranking for potential biomarkers in a cohort of samples can be accomplished by first learning the latent space representation of the multi-omic data streams (e.g., DNA and RNA and others as discussed herein), and then clustering representations and identifying a set of features (e.g., biomarkers) responsible for the largest disentanglement between sub-cohorts of interest. In accordance with various embodiments, a multi-omic unsupervised deep-learning approach (e.g., variational autoencoder) can constructed for that purpose. In accordance with various embodiments, a deep generative adversarial network can be constructed, utilizing cyclic loss between multiple data streams. In accordance with various embodiments, standard dimensionality reduction techniques (e.g., principal component analysis, individual component analysis, manifold learning) can be used to transform sparse, wide multi-omic data into a meaningful latent space. These approaches advantageously can increase power for detection of multi-omic biomarkers.

As discussed above, the systems and methods described herein, in accordance with various embodiments, can propagate ranking learned from higher levels of biological hierarchy to inform lower levels of biological hierarchy. For example, gene level ranking can inform variant lever ranking where information about occurrence of variant in various cancer annotation sources may not be available.

In accordance with various embodiments, the ranking for variants missing annotation can constructed as an aggregation of ranking for the gene and type of mutation. For example, the aggregation function is learned, which predicts the overall relevance given these aspects, after which conventional learning-to-rank algorithms can be applied to learn the ranking.

In accordance with various embodiments, clinically actionable and pathogenicity ranking can pre-loaded into the indices to increase the speed of retrieval. In accordance with various embodiments, a ranking formula learned for a specific combination of multi-omic streams can be applied at index retrieval time.

As discussed above, the systems and methods described herein, in accordance with various embodiments, can include ranking of returned results for a specific user query that can depend on a combination of multi-omic data streams queried, and can vary based on the users preferences in response to user query, taking into account clinical relevance of individual and combined multi-omic data streams.

In accordance with various embodiments, the rank can be altered by the user (e.g., returned result can be promoted or demoted). In accordance with various embodiments, the rank can be altered by indirect feedback from the user such as, for example, click rate and dwell time on a specific returned result.

As discussed above, the systems and methods described herein, in accordance with various embodiments, can provide for collecting user feedback through web-interactivity to improve multi-omic ranking of results. For example, variant, gene, pathway, derived analytics can be promoted or demoted in the list of returned results based on user feedback. In accordance with various embodiments, additional curation information can be provided and saved in the index.

In various embodiments, the systems and methods described herein can provide for an interface (or interaction with an interface) to collect explicit user feedback on relevance of returned results (e.g., user giving thumbs up/promoting/saving/saving for reporting/pinning/exporting particular result, or user giving thumbs down/demoting/deleting a result from the list of return results).

In various embodiments, the systems and methods described herein can facilitate the collection and analysis of implicit user feedback from search logs (e.g., analyzing clicks, dwelling time, query sequence, number of returned results).

In various embodiments, a collaborative search user interface can be provided (or interacted with) to allow multiple users to collaboratively refine the quality of ranking multi-omic cancer alterations (e.g., in a virtual tumor board setting).

As discussed above, the systems described herein, in accordance with various embodiments, can includes a query engine, which can be configured to perform at least one of accepting the user query, selecting, aggregating, and summarizing relevant multi-omic indices, and returning ranked multi-omic alterations for individual samples and/or cohorts of cancer samples.

In various embodiments, the query engine can be a stateless server that accepts user queries (e.g., as HTTP POST requests) and responds with a ranked list of results (e.g., as asynchronous JSON), based on a collection of pre-computed and pre-joined multi-omic index files. In various embodiments, the query engine can performs at least one of the following functions: (a) parsing the query and classifying user intent (e.g., does the user want variants, genes, pathways, samples, single sample data, cohort sample data, sample vs cohort comparison, cohort vs cohort comparison, publications, images); (b) providing query autocorrections (e.g., using autocorrection deep learning models fine-tuned on logs), providing selective synonym expansion and abbreviation expansion, generating alternative queries (e.g., using deep learning fine-tuned transformer models) and provides content-based suggestions (e.g., using fine-tuned language model for successive queries, utilizing models that take advantage of the indexed data), (c) deciding on the combination of appropriate multi-omic indices to use, (e) ranking results by their relevance to the predicted query intent (e.g., clinical relevance and pathogenicity—default ranking, frequency for some queries, amount of mutual information for others, feature weights, etc), (f) summarizing annotation documents and medical literature (e.g., using deep learning summarization techniques), and (g) handling interaction/feedback signals from the UI. In various embodiments, the query engine can allow for sub-second latency on every query and scalability to hundreds of thousands of concurrent users.

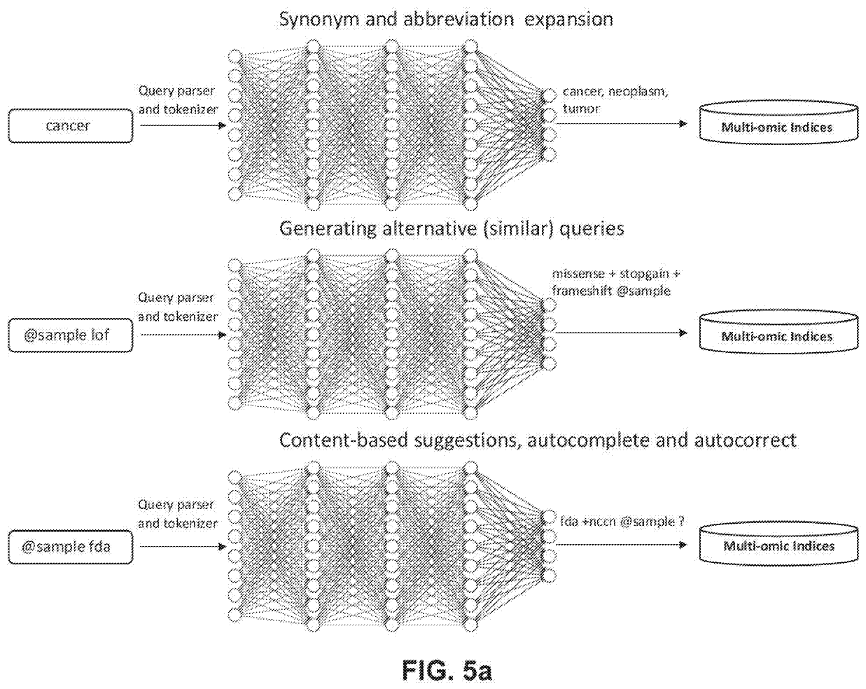

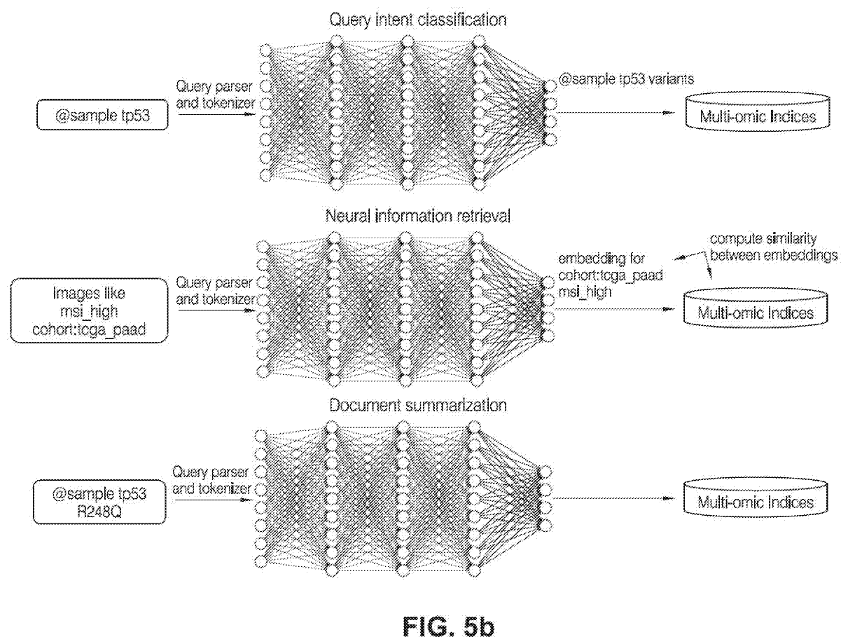

At least some of these functions are illustrated in the example workflows of FIGS. 5a and 5b, which illustrate a query engine workflow that functions to (1) produce synonym and abbreviation expansion, (2) generate alternative (similar) queries, (3) produce content-based suggestions and provide query autocompletion and autocorrection functionality, (4) classify user query intent (e.g., does the user want variants, genes, pathways, samples, single sample data, cohort sample data, sample vs cohort comparison, cohort vs cohort comparison, publications, images?), (5) perform neural information retrieval (e.g., based on a joint embedding of query and indexed documents) and (6) provide summarization of documentation (e.g., multiple sources text summarization), which can be delivered back to user via the system UI. In accordance with various embodiments, topic-specific term embeddings can be used for query expansion, particularly in (2) above. In accordance with various embodiments, for text data, the neural information retrieval model can consider both matches in the term space as well as matches in the latent space. Moreover, named entity recognition models for, for example, variants, genes, pathways, drugs, and cancer types can also be integrated to improve recall. Note that specific queries, data and summaries referenced in FIGS. 5a and 5b are exemplary only and in no way limiting to the types of queries submitted, documents analyzed, and summaries produced. For example, in the case of the specific example workflow illustrated across FIGS. 5a and 5b, given the particular parameters of that query, the query engine could conclude that while loss-of-function events in TP53 are very common in cancer, the R248 variants seem not only to result in loss of tumor-suppression, but also can act as a gain-of-function mutation that can promote tumorigenesis in mouse models (see annotation sources CIViC and Database of Genomic Biomarkers for Cancer Drugs [GDKB]).

As discussed above, the systems and methods described herein, in accordance with various embodiments, can facilitate the integrating of query term expansion using deep learning models trained on bio-medical literature and medical ontologies available (e.g., GO, UMLS, DO, MeSH, eVOC, HPO, MPO).

As discussed above, the systems described herein, in accordance with various embodiments, can facilitate the integrating of neural information retrieval models aim to provide better semantic understanding capabilities for ranking literature, images, and annotations. In various embodiments, distributed representations of words (like those generated by word2vec) can be combined to generate embeddings for queries and documents, and averaged embeddings can be used to generate effective document similarity retrieval.

An example of an effective way to do query-specific ranking is to build ranking schema for each query independently. Training models for each query separately, however, suffers from the lack of labeled data for unseen queries. However, in accordance with various embodiments, the cancer genomic alteration search engine can allow for grouping the types of queries and fine-tune ranking for specific subsets of queries of vital clinical importance (e.g., queries returning cancer alterations in the order of their clinical actionability and pathogenicity, queries returning genes in the order of their clinical actionability). To derive variant and gene clinical actionability, one can use hand labeled corpuses of queries and documents pairs. In various embodiments, precision and recall of results are measured.

In various embodiments, training corpus sets can include comprehensive cancer cases manually examined by cancer analysts.

In various embodiments, manual training corpus can be constructed by, for example, a cancer analyst/curator. The analyst/curator can examine, for example, (1) an alteration in a gene that is significantly mutated within well characterized cohort of the same cancer type (e.g., TCGA, ICGC, internal cohort) (>0.02 p or q value from MutSigCV); (2) the rank of the significantly mutated gene; (3) if mutations detected are of the same type as in well characterized cohort (e.g., missense, indel, nonsense); (4) if mutation is a missense whether it occurs at a hot spot; (5) the number of patients from the well characterized cohort with this mutation; and (6) in some cases further examination of the mutation, position, structure, and the cancer type of patient with the mutation is conducted.

As discussed above, the systems and methods described herein, in accordance with various embodiments, can provide for a universal search interface (as opposed to many different entry points). In various embodiments, all knowledge, whether it be, for example, multi-omic cancer data, samples, variants, genes, drugs, pathways, phenotypes, medical literature, image data, derived cancer analytics, machine learning models for predicting tumor characteristics and their features, upload of user data, etc., can be accessible through the same simple search interface.

As discussed above, the systems and methods described herein, in accordance with various embodiments, can provide for a checklist/terminal of key actionable and important cancer alterations, derived cancer analytics, and quality control metrics for the clinician or researcher working with either individual samples or a cohort of samples.

The systems and methods described herein, in accordance with various embodiments, can provide important cancer and inherited cancer variants reported according to ACMG guidelines.

The systems and methods described herein, in accordance with various embodiments, can provide a dynamic hyperlinked individual patient and cohort report, where at least some of the items on the report are hyperlinked to multi-modal cancer search queries, cancer alterations are ranked. In various embodiments, hyperlinked report content can be generated dynamically based on the queries that the user makes and saves for the reporting purposes.

The systems and methods described herein, in accordance with various embodiments, allows for including at least one of integrated multi-omic results, visualizations, images, medical literature, advanced cancer analytics and data from cancer bioinformatics pipelines at any level (e.g., sequencing coverage, percent of types base pair changes, visualization of sequencing reads that support an individual variant) in the dynamic reports generated by saved user queries for reporting.

The systems and methods described herein, in accordance with various embodiments, can be run as web services with two factor authentication and access control layer, to help ensure that every client has access only to the samples they are authorized to access and no analytics is being carried out across independent datasets, access to which is controlled by different entities.

In various embodiments, queries can comprise natural language terms (which can be conceptually arbitrary) combined with special operators. In various embodiments, queries can comprise speech to text models. In various embodiments, special operators can enable a user to unambiguously refer to certain information (e.g., a specific client) or impose certain constraints (e.g., provide only genes or pathways as results). In various embodiments, the operators can include, for example, a plus sign, a minus sign, an equal sign, an ampersand, an asterisk, quotation marks, parenthesis, brackets, curly braces, a back slash, a forward slash, a colon, a semi-colon, a hash sign(#), an at sign (@), a tilde sign (˜), an equals sign (=), a greater than sign (>), a less than sign (<), and words AND, OR, NOT, EXCEPT. In various embodiments, queries consist of natural language terms combined with special operators. In various embodiments, special operators can enable a user to refer unambiguously to certain information.

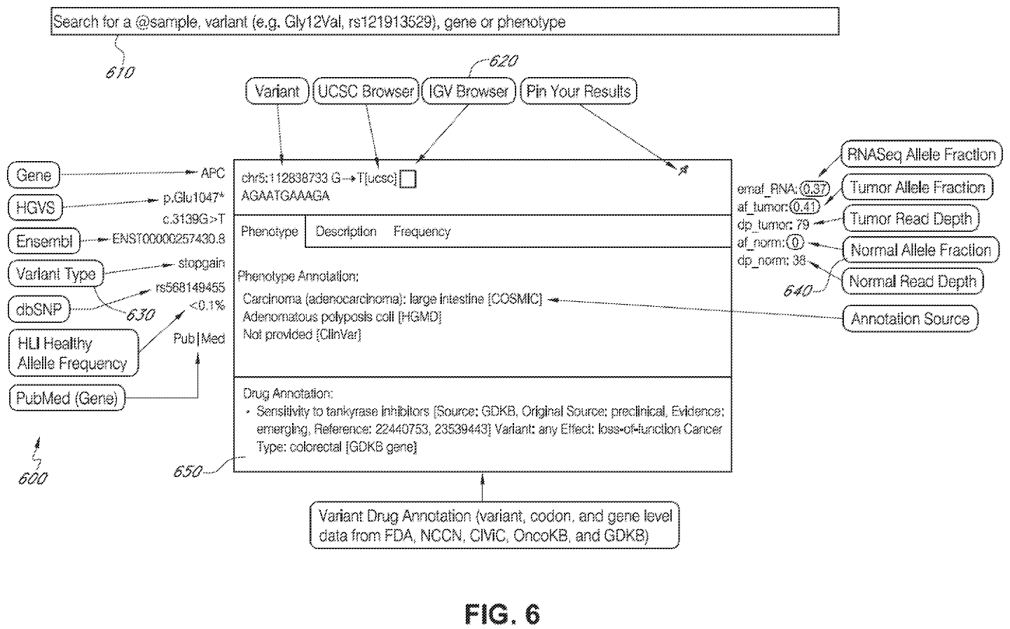

FIG. 6 illustrates an example of a user interface 600 with a single search box 610 that allows users to enter different queries and receive ranked results. Each variant can be displayed with rich data that includes, for example, variant quality control, variant metrics, allele frequency compared to population databases, therapeutic drug annotation, comparison with cancer databases and annotation sources, and the ability to view the mutation and surrounding sequencing reads using an integrated genome variant browser (IGV) and explore the variant in the UCSC genome browser.

Section 620 of UI 600 allows the user to examine the location and quality of the variant call. The chromosome, position, and variant can be listed with the mutated base highlighted in a different color than the reference. The UCSC link allows the user to view the variant in the genome browser (allowing deep investigation into the variant). The actual sequencing reads can be visualized using the IGV link, which will allow the user to, for example, determine the reliability of the variant call, see if the variant occurs in a messy region, or if the call is unreliable due to a sequencing artifact.

Section 630 of UI 600 lists gene level information. The gene name is listed and when clicked, can proceed to deep information regarding the variant, including a gene summary, the frequency of that variant in TCGA data. As such, the user can investigate if that variant is found and at what frequency in the same as well as other tumor types. Clinical trials for that variant as well as other relevant clinical information can be displayed. The HGVS tab displays the protein level variant. The Ensembl tab displays the transcript used to map the protein, and the dbSNP rsID are also listed. The variant can be compared to the frequency found in the healthy population (see “HLI Healthy Allele Frequency” in FIG. 6). The PubMed tab links out to relevant papers regarding that variant in scientific literature from PubMed.

Section 640 of UI 600 can allow the user to perform quality control of the variant call. If RNA-Seq was also performed, the RNA-Seq allele fraction is displayed. The tumor and normal allele factions and read depths allow the user to determine the call quality and if there is any evidence of the variant in normal blood.

Box 650 of UI 600 provides clinical information, if available.

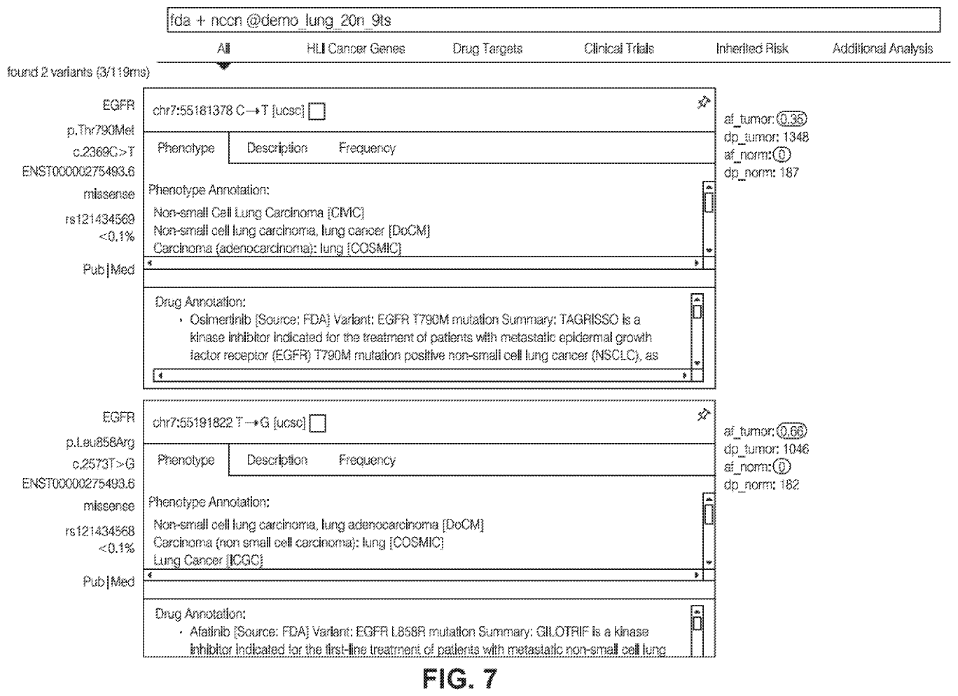

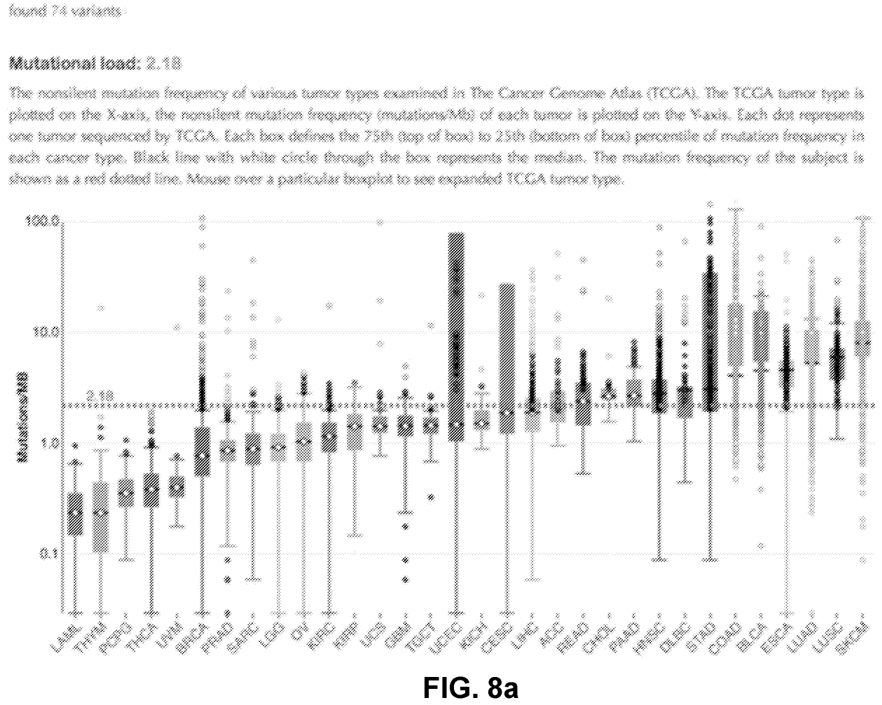

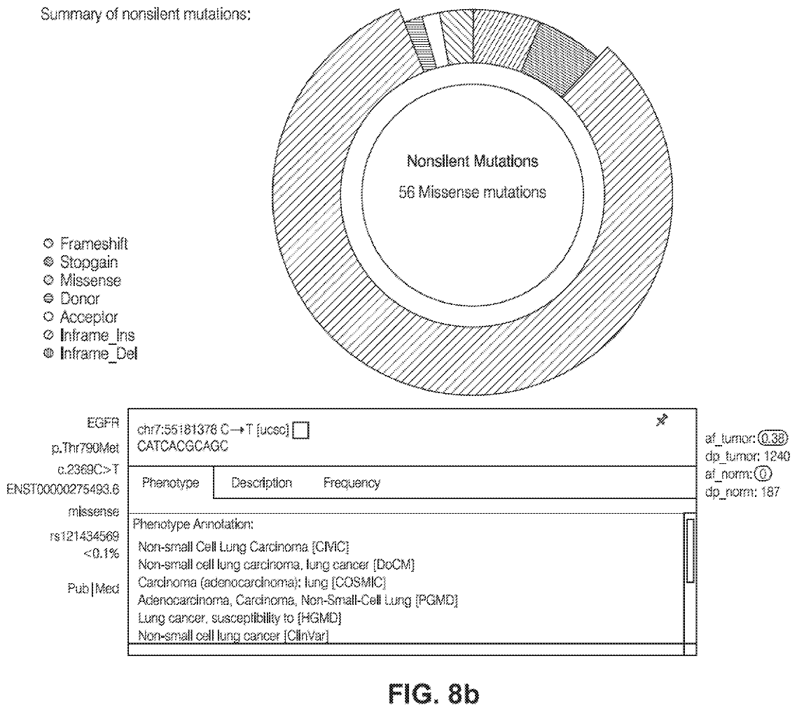

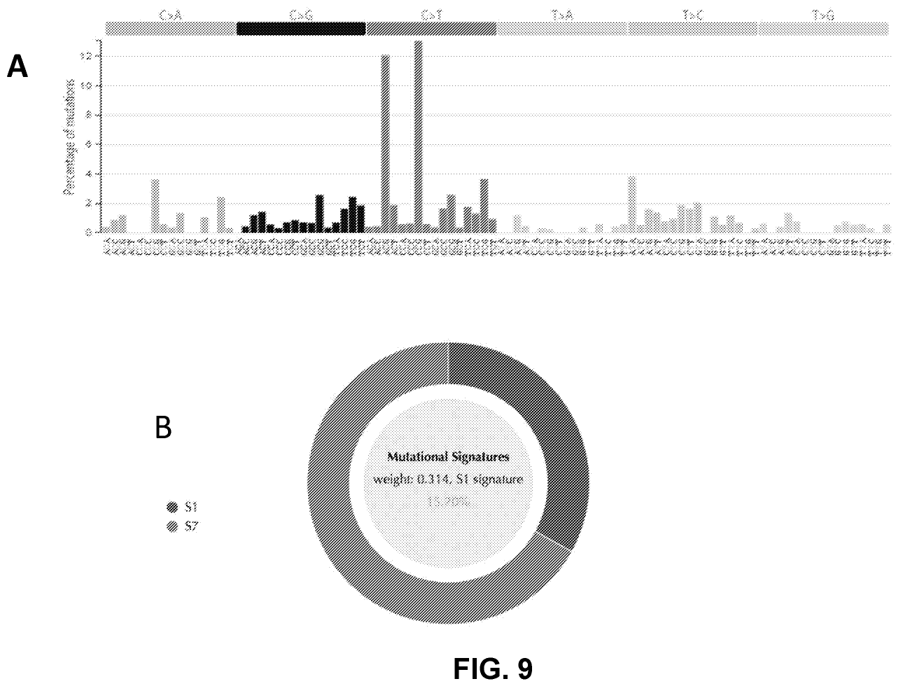

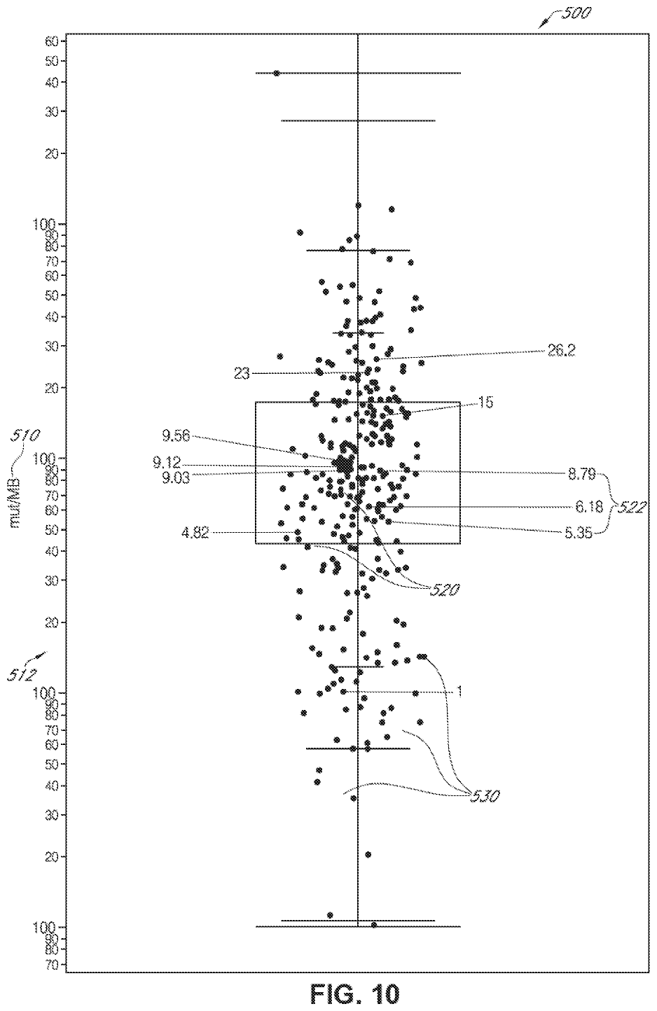

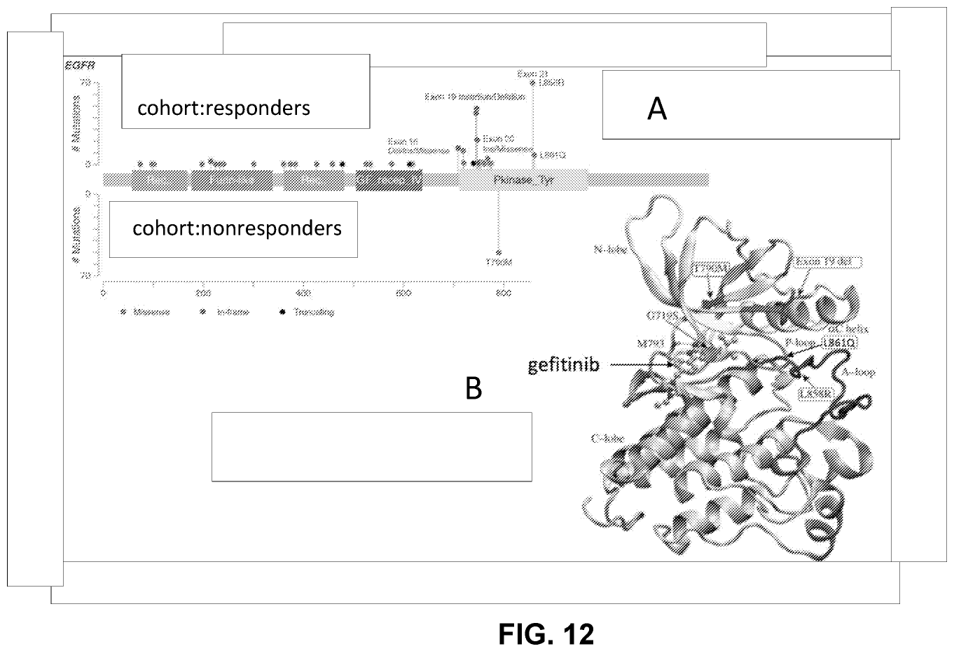

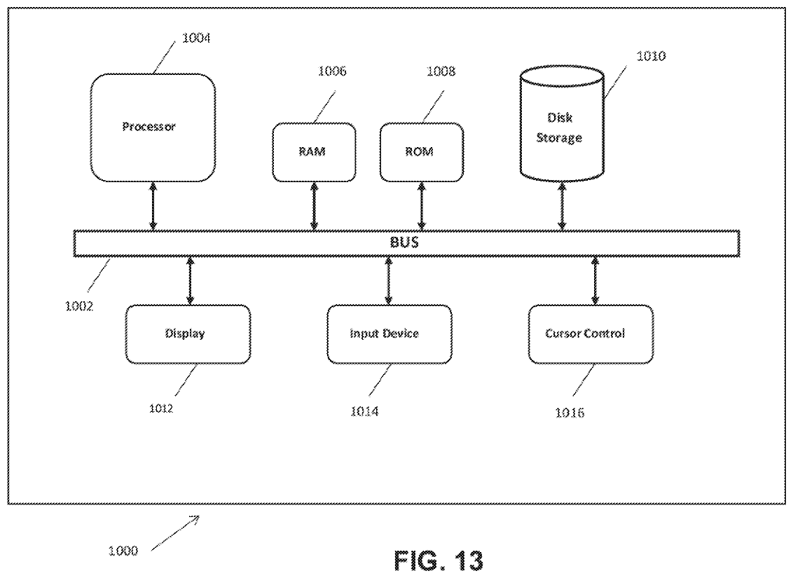

In various embodiments, the systems described herein can include an interface allowing a user to enter a user query, or use of the same. In various embodiments, the methods described herein can provide for entry of a user query via an interface, or for use of the same. As discussed above, in various embodiments, the user query can be by speech. In various embodiments, the user query can include, for example, a patient/individual ID number, a cohort name/ID number, a certain gene name or gene symbol, a particular annotation source, a variant, and/or a phenotype. In various embodiments, the input can be a check box or clickable button that restricts or filters the output to sequence, for example, variants, genes, phenotypic data, a particular combination of multi-omic data stream, and statistically significant variants, genes, pathways. In various embodiments, the results can be sortable, designated as favorite where appropriate, or exported to another program or exported to a dynamically generated report. In various embodiments, individual search terms can be combinable. In various embodiments, an individual (or user) can search within a certain set of results for additional information using additional user queries or filtering. Table 1 exemplifies a non-exhaustive list of examples of the information desired, example user input, and example output. Table 1 is not an exclusive or exhaustive list of queries that can be deployed by a user.

| Type information desired by user | Example user input | Example output |
|---|---|---|
| Patient (individual and | @ PatientSeqID person | Patient's cancer type, Sequencing depths, |
| all patients under a physician) | | selected sequencing quality metrics |
| Somatic mutations with | @ PatientSeqID fda | Ranked list of gene and/or variants with |
| FDA approved therapeutics | | annotation from FDA labels; linked out to |
| | | FDA and other annotation sources |
| Somatic mutations with FDA | fda + noun @ PatientSeqID | Ranked list of gene and/or |
| approved or professional | | variants with annotation from FDA and |
| guidelines therapeutics FIG. 7 | | NCCN; linked out to annotation sources |
| Somatic mutations | @ PatientSeqID nonsilent genes | Ranked list of gene and/or variants with |
| matching clinical trials | | clinical trials information; linked out to |
| | | clinicaltrials.gov and annotation sources |
| Somatic mutations in | @ PatientSeqID TP53 | Use all somatic variants by the TP53 gene |
| a specific cancer gene | | |
| Tumor Mutational | @ PatientSeqID afrac > 0.05 tmb | A number representing non silent mutations |
| Burden (TMB) FIG. 8 | | in the exome divide by the size of exome |
| | | (mutation/MB), overlaid on the Cancer |
| | | Genome Atlas (TOBA) cohort of |
| | | various cancer types |
| Mutation signature FIG. 9 | @ PatientSeqID mutsig | Patterns that categories the underlying |
| | | mutational processes that result |
| | | in somatic mutations |
| Microsatelite Instability | @ PatientSeqID mst | Scoring system to classify somatic insertions |
| (MSI) status | | and deletions that are located in repeated DNA |
| | | motifa called microsatelittes of the genome |
| All reinvent somatic mutations | @ PatientSeqID nonsilent | A list of marked non silent somatic mutations |
| | panel reportable afracs-0.05 | that are at or above 5% tumor allele fraction |
| | | in a defined set of cancer series |
| Inherited cancer risk variants | @ PatientSeqIDg nonsilent | A list of non silent germlike variants in a set |
| | panel:hll-inh at < 0.82 | of defined genes that are associated with |
| | | inherited cancer risks, and at variant frequency |
| | | of less thatn 2% in a reference population |
| Immune profile based | @ PatientSeqID immuno | Attributes for immunotherapy considerations, |
| on tumor RNA Seq | | a.g. TMB. 8st of nonsilent mutations, |
| | | neo-open reading frames HLA typing |
| Unique somatic variants in two | @ PatientSeqID3-@ PatientSeqID2 | Ranked list of somatic variants present |
| different tumor smples from | | in tumor 1 but not in tumor 2 |
| the same patient or different | | |
| patients with the same tumor type | | |
| Common somatic variants in two | @ PatientSeqID1 @ PatientSeqID2 | Ranked list of somatic vaiants present |
| or more different tumor samples | | in both tumor 1 and tumor 2 |
| Compare patient's somatic mutations | @ PatientSeqID1 vs cohorritaga_paad | Graphical representation of the patient's in |
| to public data of the same tumor type | | the TCGA cohort of the same tumor type |
| Gain insights on affected biological | @ PatientSeqID nonsilent pathways | Pathway enrichment analysis of biological |
| pathways based on somatic mutations | | pathways affected by the identified variants |
| Comparing Tumor Mutational Burden | @ cohort:cohortID tmb | Display of each patient's TMB (non silent |
| (TMS) of each patient in | | mutations in the exome divide by the size of |
| the cohort FIG. 10 | | enome, expressed in mutations/MB) in the cohort |
| Gene level mutation profile and | @ cohort:cohortID panelege nonsilent | Display of each patient's somatic mutations |
| comparing clinical information | | based on most frequently mutated genes or |
| of the cohort FIG. 13 | | a defined se of genes in the cohort, display of |
| | | the number and type of somatic mutations in |
| | | each patient, aligned with clinical data if avalable |
| Stratifying cohort based on | @ cohort:responders | Gene and protein projection display of each |
| somatic mutations and | cohort.nonresponders egfr | patient's somatic mutations in EGFR gene, |
| clinical information FIG. 12 | | aligned with patient's responsiveness |
| | | to a given clinical treatment |

Note that all references to Figures in Table 1 are for guidance only and not meant to limit to relative user input and example output relative to the type of information desired by user. For example, FIG. 7 illustrates an example of search results obtained with a particular syntax (“fda+nccn @PatientSeqID”), in accordance with various embodiments.

Further, for example, FIGS. 8a and 8b illustrate an example of search results obtained with a particular syntax (“@PatientSeqID afrac>0.05 tmb”), in accordance with various embodiments. FIG. 8b, in particular, illustrates a display of one of the non-silent mutations contributing to overall tumor mutation burden of the tumor in this specific example. In further detail, FIGS. 8a and 8b shows one example of search results obtained with the particular above-referenced syntax, where the user would like tumor mutation burden value only counting mutations that have allele fraction greater than 5%. Tumor mutation burden can then be displayed on the background on the Cancer Genome Atlas tumor mutation values grouped by cohort. The number of types of non-silent mutations found in the tumor sample can also be displayed in the illustrated pie chart (see FIG. 8b). This display can allow the user to quickly assess potential cancer subtypes, potential sequencing problems, as well as an overall assessment of what is behind the tumor mutation burden value. The center region of the illustrated pie chart displays the total count of non-silent mutations. The total number of non-silent mutations is further broken down into the types of non-silent mutations that have been identified, again, by referring outside the center region of the pie chart (legend provided adjacent the pie chart). In many cancers (as seen in this example), missense mutations can be the most frequent. If microsatellite instable frameshift mutations make up a large fraction of mutations, the pie chart display function allows a quick examination of that parameter. Various sequencing artifacts can also result in high percentages of mutation types that are not normally seen in that cancer. The pie chart display function can also be used to determine the clinical relevance of the tumor mutation burden. Some immunotherapy agents work best on tumors that are composed mainly of frameshift mutations or other specific mutation types. As such, the pie chart display function will allow the user to quickly assess those possibilities. Below the charts, the interface produces a ranked list of all non-silent variants with allele fraction greater than 5% is displayed (FIG. 8b displays a single hit due to lack of space).

Further, for example, FIG. 9 illustrates an example of search results returned from a user query, in accordance with various embodiments. In particular, FIG. 9. illustrates a non-limiting example of search results obtained with a particular syntax, “@PatientSeqID mutsig”. The mutational signature is the overall pattern of base pair changes that occur in the tumor across all genes. Mutational signature can be derived by counting all the base pair changes in context to arrive at an overall mutagenesis pattern. A readily used definition of mutation signatures can be found at https://cancer.sanger.ac.uk/cosmic/signatures. The identification of the mutation signature can guide treatment, can help explain the underlying causes of the tumor, and can help resolve variants of unknown significance. Mutational signature is therefore important to analyzing a tumor's overall characteristic.

Section A of FIG. 9 displays an X-Y chart of a type of base pair substitution type (i.e. C>A, C>G, C>T, T>A, T>C, T>G) in the context of the base pairs surrounding the mutation (3 bp, displayed on the X-axis). The frequency of each mutation type is plotted on the Y-axis. In this example case, the graph is compared to the COSMIC identified signature to arrive at overall mutational signatures of the tumor.

Section B displays, on a pie chart, the percentage of overall mutation signatures found in the tumor. This display can allow the user to determine the major signature in the tumor along with any minor signatures that are identified. In this example, from a melanoma tumor, the major signature displayed is S7, which is consistent with the literature. If the mutational signature displayed is not expected for that cancer type, the user can conduct further investigations.

The mutation signature can also help guide clinical decisions. For example, consider BRCA1/2 mutations in breast and ovarian cancer. PARP inhibitors may be used in BRCA1/2 mutated cases of breast and ovarian cancer. COSMIC signature 3 can be characterized by deficiencies in BRCA or pathway genes, whereby identifying signature 3 in tumors indicates a BRCA mutational process, even in the absence of an identified mutation. If the tumor contains unknown significance BRCA mutations, assaying the presence of signature 3 can help determine if the mutation is functional. In both cases, the potential benefit of PARP inhibitors may be explored.

A further function accessible here are reconstruction weights (not shown) for each of the 96 triplets.

Further, for example, FIG. 10 illustrates an example of search results returned from a user query, in accordance with various embodiments. In particular, FIG. 10 illustrates a non-limiting example of search results obtained with a particular syntax, “cohort:CohortID tmb”. The query for this case can be to identify the tumor mutational burden in the cohort. The tumor mutational burden (TMB, mutations/mb) of each tumor in the cohort (circles having associated numerical TMB values associated therewith) can be compared to the TMB of tumors from the same cancer type (in this case, pancreatic carcinoma-PAAD) by The Cancer Genome Atlas (remainder of, and the majority of, circles on the plot, with no associated TMB values referenced). The TMB is represented on the Y-axis, which allows the user to see if the TMB identified in the cohort is consistent with prior knowledge about that cancer. The TCGA median for PAAD is shown as a horizontal line in the middle of the box. The representation using box and whisker plots allows the user to see if the cohort samples plot within the average or outlier range found in TCGA.

Referring to FIG. 10, a cohort TMB chart 500 is provided, with TMB 510 represented on the Y-axis 512. The tumor mutational burden (TMB, mutations/mb) of each tumor in the cohort are first points 520 having associated numerical TMB values 522 associated therewith. Those values are compared against the TMB of tumors from the same cancer type (in this case, pancreatic carcinoma-PAAD) by The Cancer Genome Atlas represented by second points 530, which have no associated TMB values associated therewith, and which, in this example, make of the majority of captured points.

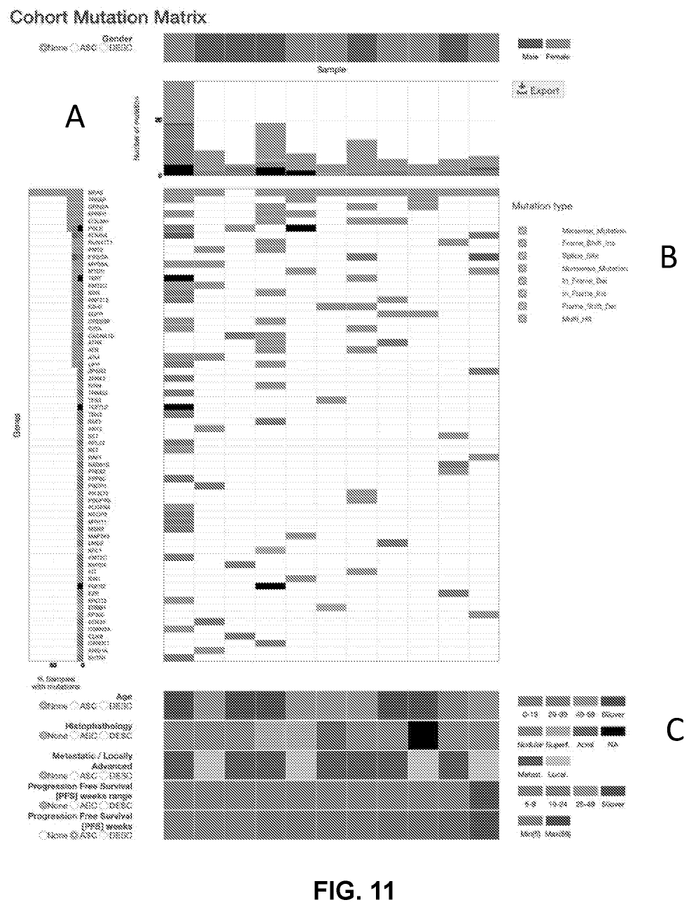

Further, for example, FIG. 11 illustrates an example of search results returned from a user query, in accordance with various embodiments. In particular, FIG. 11 displays an integrated summary of multiple genomic alterations and clinical information examined in a cohort of samples in response to user query “cohort:CohortID panel:cgc nonsilent”, asking to summarize non-silent mutations in the Cancer Gene Census panel in a particular cohort. Effectively, the query for this case can be to identify whether the samples in the given cohort have the same numbers and types of mutations. Each tumor sample can be displayed in a column, each gene in a row, and available clinical information can be added to the table. The plot can be stratified by any of the clinical parameters displayed. The plot can be initially sorted by the most frequently mutated cancer genes in the cohort (as illustrated), with the gene level frequency displayed. The mutation types (e.g., missense, nonsense, frameshift) can be identified by type of variant using different box colors (see section B of FIG. 11). In the illustrated example, the driver gene (NRAS) is a missense mutation as expected. The total mutation count for each sample can also displayed, information of which the user can use to sort the plot. The display feature can allow the user to perform deep analysis on the cohort, as well as identify specific alterations for any individual sample. The co-occurrence or mutual exclusivity of mutations can be seen in this plot. Individual mutations can be listed below the chart (not shown).

In the case illustrated in FIG. 11, section A illustrates that the sample on the far left end has the highest amount of mutations. The type of mutations are fairly consistent among this cohort. In some cases, a sample with extremely high mutation counts with high frameshift types of mutations may be observed. This observation could warrant more exploration to determine if the sample is microsatellite instable or there is an artifact. Moreover, the sample third from the left does not have the NRAS mutation that the rest of the samples do. However, the number and types of mutations are different from the rest of the cohort. This observation may necessitate more thorough exploration to determine if this difference is artifact or biological. Section C illustrates a mutation table plot that can be sorted using clinical data.

Further, for example, FIG. 12 illustrates an example of search results returned from a user query, in accordance with various embodiments. In particular, FIG. 12 shows a non-limiting example of search results obtained with a particular syntax, “cohort:responders cohort:nonresponders egfr”, where the user would like to compare mutations in a gene EGFR in two sub-cohorts: responders and non-responders. Ranked individual mutations can be listed below (not shown in this figure). In this example, section A provides an EGFR gene-level schematic of germline/somatic mutations in the two cohorts (cohort responders vs. cohort nonresponders). Section B provides a 3D protein structure, highlighting the position affected by hotspot mutations that clustered near a drug (gefitinib) binding site for the two cohorts.

FIG. 13 is a block diagram that illustrates a computer system 1000, upon which embodiments, or portions of the embodiments, of the present teachings may be implemented. In various embodiments of the present teachings, computer system 1000 can include a bus 1002 or other communication mechanism for communicating information, and a processor 1004 coupled with bus 1002 for processing information. In various embodiments, computer system 1000 can also include a memory 1006, which can be a random access memory (RAM) or other dynamic storage device, coupled to bus 1002 for determining instructions to be executed by processor 1004. Memory 1006 also can be used for storing temporary variables or other intermediate information during execution of instructions to be executed by processor 1004. In various embodiments, computer system 1000 can further include a read only memory (ROM) 1008 or other static storage device coupled to bus 1002 for storing static information and instructions for processor 1004. A storage device 1010, such as a magnetic disk or optical disk, can be provided and coupled to bus 1002 for storing information and instructions.

In various embodiments, computer system 1000 can be coupled via bus 1002 to a display 1012, such as a cathode ray tube (CRT) or liquid crystal display (LCD), for displaying information to a computer user. An input device 1014, including alphanumeric and other keys, can be coupled to bus 1002 for communicating information and command selections to processor 1004. Another type of user input device is a cursor control 1016, such as a mouse, a trackball or cursor direction keys for communicating direction information and command selections to processor 1004 and for controlling cursor movement on display 1012. This input device 1014 typically has two degrees of freedom in two axes, a first axis (i.e., x) and a second axis (i.e., y), that allows the device to specify positions in a plane. However, it should be understood that input devices 1014 allowing for 3-dimensional (x, y and z) cursor movement are also contemplated herein. The display and input device (or interface as also used herein), is discussed in greater detail herein as to capabilities beyond that which is discussed here.

Consistent with certain implementations of the present teachings, results can be provided by computer system 1000 in response to processor 1004 executing one or more sequences of one or more instructions contained in memory 1006. Such instructions can be read into memory 1006 from another computer-readable medium or computer-readable storage medium, such as storage device 1010. Execution of the sequences of instructions contained in memory 1006 can cause processor 1004 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein, and will be discussed in greater detail below, refers to any media that participates in providing instructions to processor 1004 for execution. Such a medium can take many forms, including but not limited to, non-volatile media, volatile media, and transmission media. Examples of non-volatile media can include, but are not limited to, optical, solid state, magnetic disks, such as storage device 1010. Examples of volatile media can include, but are not limited to, dynamic memory, such as memory 1006. Examples of transmission media can include, but are not limited to, coaxial cables, copper wire, and fiber optics, including the wires that comprise bus 1002.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read. Further discussion on media is provided below.

In addition to computer readable medium, instructions or data can be provided as signals on transmission media included in a communications apparatus or system to provide sequences of one or more instructions to processor 1004 of computer system 1000 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc. Further discussion on data communications is provided below.

It should be appreciated that the methodologies described herein including flow charts, diagrams and accompanying disclosure can be implemented using computer system 1000 as a standalone device or on a distributed network of shared computer processing resources such as a cloud computing network.

It should further be appreciated that in certain embodiments, machine readable storage devices are provided for storing non-transitory machine-readable instructions for executing or carrying out the methods described herein. The machine-readable instructions can control all aspects of the systems and methods described herein. Furthermore, the machine-readable instructions can be initially loaded into the memory module or accessed via the cloud or via the API.

In various embodiments, the systems and methods described herein can include a digital processing device, or use of the same. In various embodiments, the digital processing device can includes one or more hardware central processing units (CPUs) or general-purpose graphics processing units (GPGPUs) that carry out the device's functions. In various embodiments, the digital processing device further comprises an operating system configured to perform executable instructions. In various embodiments, the digital processing device can be optionally connected a computer network. In various embodiments, the digital processing device can be optionally connected to the Internet such that it accesses the World Wide Web. In various embodiments, the digital processing device can be optionally connected to a cloud computing infrastructure. In various embodiments, the digital processing device can be optionally connected to an intranet. In various embodiments, the digital processing device can be optionally connected to a data storage device.

In accordance with various embodiments, suitable digital processing devices can include, by way of non-limiting examples, server computers, desktop computers, laptop computers, notebook computers, sub-notebook computers, netbook computers, netpad computers, handheld computers, Internet appliances, mobile smartphones, tablet computers, and personal digital assistants. Those of ordinary skill in the art will recognize that many smartphones are suitable for use in the system described herein. Those of ordinary skill in the art will also recognize that select televisions, video players, and digital music players with optional computer network connectivity are suitable for use in the system described herein. Suitable tablet computers include those with booklet, slate, and convertible configurations, known to those of ordinary skill in the art.

In various embodiments, the digital processing device includes an operating system configured to perform executable instructions. The operating system can be, for example, software, including programs and data, which manages the device's hardware and provides services for execution of applications. Those of ordinary skill in the art will recognize that suitable server operating systems include, by way of non-limiting examples, FreeBSD, OpenBSD, Net-BSD, Linux, Apple® Mac OS X Server®, Oracle® Solaris®, Windows Server®, and Novell® NetWare®. Those of ordinary skill in the art will recognize that suitable personal computer operating systems include, by way of non-limiting examples, Microsoft® Windows®, Apple® Mac OS X®, UNIX®, and UNIX-like operating systems such as GNU/Linux®. In various embodiments, the operating system is provided by cloud computing. Those of ordinary skill in the art will also recognize that suitable mobile smart phone operating systems include, by way of non-limiting examples, Nokia® Symbian® OS, Apple® iOS®, Research In Motion® Black-Berry OS®, Google® Android®, Microsoft® Windows Phone® OS, Microsoft® Windows Mobile® OS, Linux®, and Palm® WebOS®.

In various embodiments, the device includes a storage and/or memory device. The storage and/or memory device is one or more physical apparatuses used to store data or programs on a temporary or permanent basis. In various embodiments, the device is volatile memory and requires power to maintain stored information. In various embodiments, the device is non-volatile memory and retains stored information when the digital processing device is not powered. In various embodiments, the non-volatile memory comprises flash memory. In some embodiments, the non-volatile memory comprises dynamic random-access memory (DRAM). In various embodiments, the non-volatile memory comprises ferroelectric random access memory (FRAM). In various embodiments, the non-volatile memory comprises phase-change random access memory (PRAM). In various embodiments, the device is a storage device including, by way of non-limiting examples, CD-ROMs, DVDs, flash memory devices, magnetic disk drives, magnetic tapes drives, optical disk drives, and cloud computing based storage. In various embodiments, the storage and/or memory device is a combination of devices such as those disclosed herein.

In various embodiments, the digital processing device includes a display to send visual information to a user. In various embodiments, the display is a cathode ray tube (CRT). In various embodiments, the display is a liquid crystal display (LCD). In various embodiments, the display is a thin film transistor liquid crystal display (TFT-LCD). In various embodiments, the display is an organic light emitting diode (OLED) display. In various embodiments, on OLED display is a passive-matrix OLED (PMOLED) or active-matrix OLED (AMOLED) display. In various embodiments, the display is a plasma display. In various embodiments, the display is a video projector. In various embodiments, the display is a combination of devices such as those disclosed herein.

In various embodiments, the digital processing device includes an input device to receive information from a user. In various embodiments, the input device is a keyboard. In various embodiments, the input device is a pointing device including, by way of non-limiting examples, a mouse, trackball, track pad, joystick, game controller, or stylus. In various embodiments, the input device is a touch screen or a multi-touch screen. In various embodiments, the input device is a microphone to capture voice or other sound input. In various embodiments, the input device is a video camera or other sensor to capture motion or visual input. In various embodiments, the input device is a Kinect, Leap Motion or the like. In various embodiments, the input device is a combination of devices such as those disclosed herein.

In various embodiments, the systems disclosed herein can include, and the methods herein can be run on, one or more non-transitory computer readable storage media encoded with a program including instructions executable by the operating system of an optionally networked digital processing device. In various embodiments, a computer readable storage medium is a tangible component of a digital processing device. In various embodiments, a computer readable storage medium is optionally removable from a digital processing device. In various embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In various embodiments, the program and instructions are permanently, substantially permanently, semi-permanently, or non-transitorily encoded on the media.

In various embodiments, the systems and methods disclosed herein can include at least one computer program, or use at least one computer program. A computer program includes a sequence of instructions, executable in the digital processing device's CPU, written to perform a specified task. Computer readable instructions may be implemented as program modules, such as functions, objects, Application Programming Interfaces (APis), data structures, and the like, that perform particular tasks or implement particular abstract data types. Those of ordinary skill in the art will recognize that a computer program may be written in various versions of various languages.

The functionality of the computer readable instructions may be combined or distributed as desired in various environments. In various embodiments, a computer program comprises one sequence of instructions. In various embodiments, a computer program comprises a plurality of sequences of instructions. In various embodiments, a computer program is provided from one location. In various embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

In various embodiments, a computer program includes a web application. Those of ordinary skill in the art will recognize that a web application, in various embodiments, utilizes one or more software frameworks and one or more database systems. In various embodiments, a web application is created upon a software framework such as Microsoft® .NET or Ruby on Rails (RoR). In various embodiments, a web application utilizes one or more database systems including, by way of non-limiting examples, relational, non-relational, object oriented, associative, and XML database systems. In various embodiments, suitable relational database systems include, by way of non-limiting examples, Microsoft® SQL Server, mySQL™, and Oracle®. Those of ordinary skill in the art will also recognize that a web application, in various embodiments, is written in one or more versions of one or more languages. A web application may be written in one or more markup languages, presentation definition languages, client-side scripting languages, server-side coding languages, data-base query languages, or combinations thereof. In various embodiments, a web application is written to some extent in a markup language such as Hypertext Markup Language (HTML), Extensible Hypertext Markup Language (XHTML), or eXtensible Markup Language (XML). In various embodiments, a web application is written to some extent in a presentation definition language such as Cascading Style Sheets (CSS). In various embodiments, a web application is written to some extent in a client-side scripting language such as Asynchronous Javascript and XML (AJAX), Flash® Actionscript, Javascript, or Silverlight®. In various embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tel, Smalltalk, WebDNA®, or Groovy. In various embodiments, a web application is written to some extent in a database query language such as Structured Query Language (SQL). In various embodiments, a web application integrates enterprise server products such as IBM® Lotus Domino®. In various embodiments, a web application includes a media player element. In various embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

In various embodiments, a computer program includes a mobile application provided to a mobile digital processing device. In various embodiments, the mobile application is provided to a mobile digital processing device at the time it is manufactured. In various embodiments, the mobile application is provided to a mobile digital processing device via the computer network described herein.

A mobile application can be created by techniques known to those of ordinary skill in the art using hardware, languages, and development environments known to the art. Those of ordinary skill in the art will recognize that mobile applications can be written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelera-tor®, Celsius, Bedrock, Flash Lite, .NET Compact Frame-work, Rhomobile, and WorkLight Mobile Platform. Other development environments are available without cost including, by way of non-limiting examples, Lazarus, Mobi-Flex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of ordinary skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo DSi Shop.

In various embodiments, a computer program includes a standalone application, which is a program that is run as an independent computer process, not an add-on to an existing process, e.g., not a plug-in. Those of ordinary skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often per-formed, at least in part, to create an executable program. In various embodiments, a computer program includes one or more executable complied applications.

In various embodiments, the computer program includes a web browser plug-in (e.g., extension, etc.). In computing, a plug-in is one or more software components that add specific functionality to a larger software application. Makers of software applications support plug-ins to enable third-party developers to create abilities, which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of ordinary skill in the art will be familiar with several web browser plug-ins including, Adobe® Flash® Player, Microsoft® Silver-light®, and Apple® QuickTime®. In various embodiments, the toolbar comprises one or more web browser extensions, add-ins, or add-ons. In various embodiments, the toolbar comprises one or more explorer bars, tool bands, or desk bands.

Those of ordinary skill in the art will recognize that several plug-in frame works are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Soft-ware® Opera®, and KDE Konqueror. In various embodiments, the web browser is a mobile web browser. Mobile web browsers (also called mircrobrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

In various embodiments, the systems and methods disclosed herein include a software, server and/or database modules, or incorporate use of the same in methods according to various embodiments disclosed herein. Software modules can be created by techniques known to those of ordinary skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In various embodiments, software modules are in one computer program or application. In various embodiments, software modules are in more than one computer program or application. In various embodiments, software modules are hosted on one machine. In various embodiments, software modules are hosted on more than one machine. In various embodiments, software modules are hosted on cloud computing platforms. In various embodiments, software modules are hosted on one or more machines in one location. In various embodiments, software modules are hosted on one or more machines in more than one location.

In various embodiments, the systems and methods disclosed herein include one or more databases, or incorporate use of the same in methods according to various embodiments disclosed herein. Those of ordinary skill in the art will recognize that many databases are suitable for storage and retrieval of user, query, token, and result information. In various embodiments, suitable databases include, by way of non-limiting examples, relational databases, non-relational databases, object oriented databases, object databases, entity-relationship model databases, associative databases, and XML databases. Further non-limiting examples include SQL, Postgr-eSQL, MySQL, Oracle, DB2, and Sybase. In various embodiments, a database is internet-based. In further Web. Suitable web browsers include, by way of non-limiting examples, Microsoft® Internet Explorer®, Mozilla® Fire-fox®, Google® Chrome, Apple® Safari®, Opera Soft-ware® Opera®, and KDE Konqueror. In various embodiments, the web browser is a mobile web browser. Mobile web browsers (also called microbrowsers, mini-browsers, and wireless browsers) are designed for use on mobile digital processing devices including, by way of non-limiting examples, handheld computers, tablet computers, netbook computers, subnotebook computers, smartphones, and personal digital assistants (PDAs). Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony PSP™ browser.

In various embodiments, a database is web-based. In various embodiments, a database is cloud computing-based. In other embodiments, a database is based on one or more local computer storage devices.

In various embodiments, the systems and methods disclosed herein include one or features to prevent unauthorized access. The security measures can, for example, secure a user's data. In various embodiments, data is encrypted. In various embodiments, access to the system requires multi-factor authentication and access control layer. In various embodiments, access to the system requires two-step authentication (e.g., web-based interface). In various embodiments, two-step authentication requires a user to input an access code sent to a user's e-mail or cell phone in addition to a username and password. In some instances, a user is locked out of an account after failing to input a proper username and password. The systems and methods disclosed herein can, in various embodiments, also include a mechanism for protecting the anonymity of users' genomes and of their searches across any genomes.

The systems and methods described herein, in various embodiments, can assist oncologists in deriving clinical insights during case review, or in a collaborative setting during virtual tumor boards by allowing to explore data for a patient or a set of patients at any level of the cancer bioinformatics pipeline, verify which cancer alterations are real and do not represent sequencing artifacts, report quality control values, integrate multi-omic data streams and advanced analytics to provide key dashboard or ‘not to miss’ checklist of cancer characteristics and findings and to provide clinical, prognostic, diagnostic and therapeutic information for each ranked result returned. In various embodiments, the multi-omic cancer search described herein provides “augmented intelligence” to the physician to help with clinical decisions.

Usage of the systems and methods described herein, in accordance various embodiments, can include clinicians as users. These users can use the systems and methods described herein to perform comprehensive reporting of drug targets and key alterations in tumor (and normal) genomes.

The systems and methods described herein, in accordance various embodiments, can be used in virtual tumor boards. The systems and methods described herein, in accordance various embodiments, can be used by individual clinicians as a checklist for not-to-miss important tumor properties and check on the clinical trials available within the oncologist's institution or globally. The systems and methods described herein, in accordance various embodiments, can be used by an oncologist during patient-oncologist visit conversations. In various embodiments, multiple clinicians can use collaborative functions of querying, visualizing, re-ranking clinically actionable and pathogenic cancer alterations, help to navigate available phenotypic, and imaging and literature data during virtual molecular tumor board to decide on the best diagnosis and treatment. Some of the non-limiting examples of questions the systems and methods described herein can address include, what are the clinically relevant cancer variants? are there potential therapeutics (FDA approved, NCNN, clinical trials)? is the mutation identified in the tumor real? is it supported by the high quality sequencing reads? is the mutation in a hard to sequence region? is it only present in tumor and Not in normal? is it expressed in RNA? is this mutation functional? what are the global tumor properties, tumor mutation burden or micro satellite instability? The systems can display multiple metrics that can be used to determine both the overall quality, and quality of a single variant. The systems and methods, in accordance with various embodiments, can provide for comparing the mutations of the patient to what has been previously described in public data sets such, for example, as the Cancer Genome Atlas (TCGA). The systems and methods, in accordance with various embodiments, can provide for comparing multiple biopsies for the same patient.

In various embodiments, users of the system and methods described herein can include biopharmaceutical or academic researcher, who can then perform, for example, cohort tumor profiling to characterize genetic profiles for patients with good/bad prognosis, responders/non-responders, perform quality control check, identification of drug targets, stratify cohorts on a potential drug response biomarker, and quick and iterative hypothesis generation before running more extensive analysis on additional validation or test cohorts. In various embodiments, ranked biomarkers that can stratify a cohort, their statistical significance, and their summary visualization, are returned by the system. In various embodiments, validation queries can be suggested by the search engine to perform robust algorithmic and statistical validation. In various embodiments, the system can auto-suggest iterative hypothesis refinement via proposed query refinement.

In various embodiments, the system and methods described herein can, for example, identify proteins, pathways, mutational processes that correlate with survival, resistance, response; deep dive into any differences found in one group; compare to other data sets; examine the cohort quality control to insure the cohort analytics are reliable and not skewed based on one of the quality control parameters; investigate any unusual results to insure they are not due to a systematic issue; drill down to an individual sample, an outlier or unusual result to insure it is a real result; explore further and get quick statistical significance of the analysis; perform multi-target data exploration; and search literature and annotation sources for potential therapeutics. Standard bioinformatics analyses generally do not give an ability to interactively query the data and refine a hypothesis using the domain knowledge. Internal systems are usually based on database systems and not search indexing (such as that which is discussed herein), which is able to provide relevance ranking, perform integration of multiple streams of information (e.g., genomic, transcriptomic, annotation, literature), and include built-in machine learning models of relevance.

As discussed above, the systems and methods described herein, in accordance with various embodiments, can be configured to provide a dynamic hyperlinked individual patient and cohort variant report, where all the items on the report are hyperlinked to multi-modal cancer search queries. In various embodiments, hyperlinked report content is generated dynamically based on the queries that the user makes and highlights and saves for the reporting purposes.

As discussed above, the systems and methods described herein, in accordance with variants embodiments, can be configured to possess expert review capabilities giving user ability to choose which query results are used for hyperlinked live report generation.

In various embodiments, dynamic reports are never out of date, and are updated based on newly indexed information. Moreover, the user can be notified of any new annotations, drugs, clinical trials available.

In various embodiments, the systems and methods provided herein can allows analysis to extend beyond both static clinical reports and pre-computed cancer portal analyses to offer dynamic generation of hyperlinked report for individual patient or a cohort. Examples for such reports include, but not limited to, tumor profiling, drug and trial matching, and immuno reports for individual samples, and cohort profiling reports for cohorts of samples. Reports can be tailored based on user queries, and in various embodiments, contain user pre-selected results returned by the multi-omic cancer search.

Applicant has advantageously discovered that the dynamic reporting paradigm based on multi-omic cancer search system can provide for (1) user interaction with the data beyond the capabilities of standard static PDF reports that cannot be modified or updated after extensive bioinformatics pipelines have been run; (2) ranking all the multi-omic cancer alterations in terms of their clinical actionability, pathogenicity, feature weight, or frequency; (3) user interrogation of output of the pipeline at any level form BAMs to VCFs to outputs for more complex analyses; (4) user view of not only machine learning models predictions, but also a list of ranked features that guided a particular prediction.

What is claimed is:

1. A method for utilizing multi-omic data indices for tumor profiling, the method comprising:
storing a plurality of multi-omic data indices, wherein each of the plurality of multi-omic data indices comprises cancer-specific tokenized data;
ingesting additional multi-omic data and any annotations associated with the additional multi-omic data, the additional multi-omic data related to one or more indices;
indexing the ingested additional multi-omic data and annotations while preserving gene names, gene variant names and multi-omic mapping between different data streams for the same patient in the specific index, to produce tokenized ingested additional multi-omic data;
receiving a user query;
selecting one or more relevant multi-omic data indices based on the user query;
ranking the selected one or more multi-omic data indices based on at least one of clinical actionability, pathogenicity, feature weight, or frequency, and
returning the ranked one or more multi-omic data indices to the user.

2. The method of claim 1, wherein the multi-omic data is selected from the group consisting of genomic, transcriptomic, epigenetic, chromatin accessibility, microbiomic, proteomic, phenotypic, image, relevant literature, integrated multi-omic data, and combinations thereof.

3. The method of claim 1, wherein the plurality of multi-omic data indices further comprises somatic genomic alterations, normal genomic alterations, and cancer annotation sources.

4. The method of claim 1, further comprising deriving cancer analytics for the selected one or more multi-omic data indices, wherein the cancer analytics comprises tumor characteristics selected from the group consisting of quality control, tumor mutation burden, genomic mutation signatures, microsatellite instability status, neo-antigens, HLA-allele typing, RNA confirmed variants, copy number variants, structural variants, non-coding regulatory variants, gene fusions, pathway enrichment, cancer driver identification, mutation summary, differential gene expression, immune signatures, matching information about treatment outcomes for similar patients and combinations thereof.

5. The method of claim 4, wherein the cancer analytics are derived for an individual sample or a cohort of samples.

6. The method of claim 4, wherein the cancer analytics comprises machine learning predictions and ranked features.

7. The method of claim 6, wherein the machine learning predictions are selected from the group consisting of a primary site of origin classifier, a prediction of future metastasis site classifier, prediction of microsatellite instability status, prediction of neo-antigen binding affinities, disease state stratification, determining cancer lineages, and combinations thereof.

8. The method of claim 1, further comprising propagating annotations from higher levels of genomic hierarchy to lower levels of genomic hierarchy.

9. The method of claim 1, further comprising ranking the selected one or more multi-omic data indices from higher levels of genomic hierarchy to lower levels of genomic hierarchy.

10. The method of claim 1, wherein the ranking comprises a clinical and pathogenic ranking for cancer variants and genes.

11. The method of claim 1, wherein the ranking comprises stratifying a cohort by incorporating a latent space representation for cancer data.

12. The method of claim 11, wherein the cohort is stratified into responders and non-responders.

13. The method of claim 11, wherein the cohort is stratified into long-progression free survival time and short-progression free survival time.

14. The method of claim 11, wherein the cohort is stratified into different sub-types of cancer.

15. The method of claim 11, wherein the latent space representation is performed by a neural network.

16. The method of claim 11, wherein the latent space representation is performed by dimensionality reduction techniques.

17. The method of claim 16, wherein the neural network is selected from the group consisting of autoencoders, variational autoencoders, deep belief networks, restricted Boltzman machines, feed forward, convolutional, recurrent, gated recurrent, long short-term memory, residual, and generative adversarial networks.

18. The method of claim 1, wherein the ranking further comprises a model for learning to rank selected from the group consisting of support vector machines, boosted decision trees, regression methods, neural networks, and combinations thereof.

19. The method of claim 1, wherein the ranking further comprises a deep learning ranking.

20. The method of claim 19, wherein the deep learning ranking is derived from a deep learning model selected from the group consisting of a deep semantic similarity model, convolutional deep semantic similarity model, recurrent deep semantic similarity model, deep relevance matching model, a deep and wide model, a deep language model, a transformer network, a long short-term memory network, a learned deep learning text embedding, a learned named entity recognition, Siamese neural network, interaction Siamese network, lexical and semantic matching network, and combinations thereof.

21. The method of claim 1, wherein the multi-omic data is selected from the group consisting of somatic calls from whole genome sequence data, somatic calls from whole exome sequence data, somatic panel sequencing from fresh frozen tissue, somatic panel sequencing from formalin-fixed paraffin-embedded tissue, somatic panel sequencing from liquid biopsy, tumor and normal variant calls, tumor/normal transcriptomic data indexed as variant confirmed in RNA or gene expression level, epigenetic data, chromatin accessibility data, microbiomic data, proteomic data, single cell sequencing data, and combinations thereof.

22. The method of claim 1, wherein the multi-omic data indices further comprise extracted phenotype data.

23. The method of claim 22, wherein the phenotype data is selected from the group consisting of electronic health records, clinical data, functional data, and combinations thereof.

24. The method of claim 1, wherein the multi-omic data indices further comprise featurized imaging data.

25. The method of claim 24, wherein the featurized imaging data is selected from the group consisting of histology slides, MRI images, X-rays, mammograms, ultrasounds, PET images, CT scans, and combinations thereof.

26. The method of claim 4, wherein the cancer analytics are dynamically computed after receipt of the user query.

27. The method of claim 1, wherein the indexing of the ingested additional multi-omic data and annotation further comprises indexing derived data selected from the group consisting of cancer analytics, annotations, features extracted from imaging data, phenotypic, medical literature data and its embeddings, and combinations thereof.

28. The method of claim 1, wherein the ranking further comprises matching sample alterations with established drug target labels and available clinical trials.

29. The method of claim 1, wherein the ranking further comprises cancer drug target identification in cohorts by detecting a potential biomarker that stratifies the cohort based on a clinical variable of interest and/or statistical significance, and wherein the returning the ranked one or more multi-omic data indices to the user comprises a stratification visualization.

30. The method of claim 1, wherein the returning the ranked one or more multi-omic data indices to the user further comprises a dynamic creation of hyper-linked reports for individual patients and/or cohorts that provide comprehensive profiling of a tumor.

31. The method of claim 1, wherein the user query can comprise user-uploaded data selected from the group consisting of a panel of variants, genes, pathways, disease state conditions, phenotypes of interest, and wherein the selecting comprises querying individual sample or cohort data sub-selected by the uploaded data.

32. The method of claim 1, wherein the user query can be provided via a user interface, and can comprise uploading data for indexing selected from the group consisting of genomic data, transcriptomic data, epigenetic data, chromatin accessibility data, microbiomic data, proteomic data, phenotypic data, annotation data, and combinations thereof.

33. The method of claim 1, further comprising normalizing and/or expanding the user query, classifying the intent of the query, summarizing retrieved documents, and performing document retrieval based on the similarity between the query and a document in a latent space using deep learning methods.

34. The method of claim 1, wherein at least one of the indexing, selecting and ranking comprises utilizing deep neural networks.

35. The method of claim 4, wherein deriving the cancer analytics comprises utilizing deep neural networks.

36. The method of claim 1, wherein the returning the ranked one or more multi-omic data indices to the user further comprises returning a summary visualization of the returned results along with the list of ranked results.

37. A non-transitory computer-readable medium in which a program is stored for causing a computer to perform a method for utilizing multi-omic data indices for tumor profiling, the method comprising
storing a plurality of multi-omic data indices, wherein each of the plurality of multi-omic data indices comprises cancer-specific tokenized data;
ingesting additional multi-omic data and any annotation associated with the additional multi-omic data, the additional multi-omic data related to one or more indices;
indexing the ingested additional multi-omic data and annotation while preserving gene names, gene variant names and multi-omic mapping between different data streams for the same patient in the specific index, to produce tokenized ingested additional multi-omic data;
receiving a user query;
selecting one or more relevant multi-omic data indices based on the user query;
ranking the selected one or more multi-omic data indices based on at least one of clinical actionability, and
returning the ranked one or more multi-omic data indices to the user.

38. The method of claim 37, wherein the multi-omic data is selected from the group consisting of genomic, transcriptomic, epigenetic, chromatin accessibility, microbiomic, proteomic, phenotypic, image, relevant literature, integrated multi-omic data, and combinations thereof.

39. The method of claim 37, wherein the plurality of multi-omic data indices further comprises somatic genomic alterations, normal genomic alterations, and cancer annotation sources.

40. The method of claim 37, further comprising deriving cancer analytics for the selected one or more multi-omic data indices, wherein the cancer analytics comprises tumor characteristics selected from the group consisting of quality control, tumor mutation burden, genomic mutation signatures, microsatellite instability status, neo-antigens, HLA-allele typing, RNA confirmed variants, copy number variants, structural variants, non-coding regulatory variants, gene fusions, pathway enrichment, cancer driver identification, mutation summary, differential gene expression, immune signatures, matching information about treatment outcomes for similar patients and combinations thereof.

41. The method of claim 40, wherein the cancer analytics are derived for an individual sample or a cohort of samples.

42. The method of claim 40, wherein the cancer analytics comprises machine learning predictions and ranked features.

43. The method of claim 42, wherein the machine learning predictions are selected from the group consisting of a primary site of origin classifier, a prediction of future metastasis site classifier, prediction of microsatellite instability status, prediction of neo-antigen binding affinities, disease state stratification, determining cancer lineages, and combinations thereof.

44. The method of claim 37, further comprising propagating annotations from higher levels of genomic hierarchy to lower levels of genomic hierarchy.

45. The method of claim 37, further comprising ranking the selected one or more multi-omic data indices from higher levels of genomic hierarchy to lower levels of genomic hierarchy.

46. The method of claim 37, wherein the ranking comprises a clinical ranking for cancer variants and genes.

47. The method of claim 3375, wherein the ranking comprises stratifying a cohort by incorporating a latent space representation for cancer data.

48. The method of claim 47, wherein the cohort is stratified into responders and non-responders.

49. The method of claim 47, wherein the cohort is stratified into long-progression free survival time and short-progression free survival time.

50. The method of claim 47, wherein the latent space representation is performed by a neural network.

51. The method of claim 50, wherein the neural network is selected from the group consisting of autoencoders, variational autoencoders, deep belief networks, restricted Boltzman machines, feed forward networks, convolutional networks, recurrent networks, long short-term memory networks, and generative adversarial networks.

52. The method of claim 37, wherein the ranking further comprises a model for learning to rank selected from the group consisting of support vector machines, boosted decision trees, regression models, neural networks, and combinations thereof.

53. The method of claim 37, wherein the ranking further comprises a deep learning ranking.

54. The method of claim 53, wherein the deep learning ranking is derived from a deep learning model selected from the group consisting of a deep semantic similarity model, a deep and wide model, a deep language model, a learned deep learning text embedding, a learned named entity recognition, Siamese neural network, and combinations thereof.

55. The method of claim 37, wherein the multi-omic data is selected from the group consisting of somatic calls from whole genome sequence data, somatic calls from whole exome sequence data, somatic panel sequencing from fresh frozen tissue, somatic panel sequencing from formalin-fixed paraffin-embedded tissue, somatic panel sequencing from liquid biopsy, tumor and normal variant calls, tumor/normal transcriptomic data indexed as variant confirmed in RNA or gene expression level, epigenetic data, chromatin accessibility data, microbiomic data, proteomic data, single cell sequencing data, and combinations thereof.

56. The method of claim 37, wherein the multi-omic data indices further comprise extracted phenotype data.

57. The method of claim 56, wherein the phenotype data is selected from the group consisting of electronic health records, clinical data, functional data, and combinations thereof.

58. The method of claim 37, wherein the multi-omic data indices further comprise featurized imaging data.

59. The method of claim 58, wherein the featurized imaging data is selected from the group consisting of histology slides, MRI images, X-rays, mammograms, ultrasounds, PET images, CT scans, and combinations thereof.

60. The method of claim 40, wherein the cancer analytics are dynamically computed after receipt of the user query.

61. The method of claim 37, wherein the indexing of the ingested additional multi-omic data and annotation further comprises indexing derived data selected from the group consisting of cancer analytics, annotations, features extracted from imaging data, phenotypic, medical literature data and its embeddings, and combinations thereof.

62. The method of claim 37, wherein the ranking further comprises matching sample alterations with established drug target labels and available clinical trials.

63. The method of claim 37, wherein the ranking further comprises cancer drug target identification in cohorts by detecting a potential biomarker that stratifies the cohort based on a clinical variable of interest and/or statistical significance, and wherein the returning the ranked one or more multi-omic data indices to the user comprises a stratification visualization.

64. The method of claim 37, wherein the returning the ranked one or more multi-omic data indices to the user further comprises a dynamic creation of hyper-linked reports for individual patients and/or cohorts that provide comprehensive profiling of a tumor.

65. The method of claim 37, wherein the user query can comprise user-uploaded data selected from the group consisting of a panel of variants, genes, pathways, disease state conditions, phenotypes of interest, and wherein the selecting comprises querying individual sample or cohort data sub-selected by the uploaded data.

66. The method of claim 37, wherein the user query can be provided via a user interface, and can comprise uploading data for indexing selected from the group consisting of genomic data, transcriptomic data, epigenetic data, chromatin accessibility data, microbiomic data, proteomic data, phenotypic data, annotation data, and combinations thereof.

67. The method of claim 37, further comprising normalizing and/or expanding the user query, classifying the intent of the query, summarizing retrieved documents, and performing document retrieval based on the similarity between the query and a document in a latent space using deep learning methods.

68. The method of claim 37, wherein at least one of the indexing, selecting and ranking comprises utilizing deep neural networks.

69. The method of claim 40, wherein deriving the cancer analytics comprises utilizing deep neural networks.

70. The method of claim 37, wherein the returning the ranked one or more multi-omic data indices to the user further comprises returning a summary visualization of the returned results along with the list of ranked results.

71. A system for utilizing multi-omic data indices for tumor profiling, the system comprising
an indexing unit comprising:
a storage element configured to store a plurality of multi-omic data indices, wherein each of the plurality of multi-omic data indices comprises cancer-specific tokenized data, and
an indexing engine configured to
ingest additional multi-omic data and any annotation associated with the additional multi-omic data, the additional multi-omic data related to one or more indices, and
index the ingested additional multi-omic data and annotation while preserving gene names, gene variant names and multi-omic mapping between different data streams for the same patient in the specific index, to produce tokenized ingested additional multi-omic data;
a user interface configured to receive a user query;
a query engine configured to select one or more relevant multi-omic data indices from the indexing unit based on the user query; and
a ranking engine configured to receive the selected one or more relevant multi-omic data indices, to rank the selected one or more multi-omic data indices based on at least one of clinical actionability, pathogenicity, feature weight, or frequency, and return the ranked one or more multi-omic data indices to the user via the user interface.

72. The system of claim 71, wherein the multi-omic data is selected from the group consisting of genomic, transcriptomic, epigenetic, chromatin accessibility, microbiomic, proteomic, phenotypic, image, relevant literature, integrated multi-omic data, and combinations thereof.

73. The system of claim 71, wherein the plurality of multi-omic data indices further comprises somatic genomic alterations, normal genomic alterations, and cancer annotation sources.

74. The system of claim 71, further comprising a cancer analytics engine configured to derive cancer analytics for the selected one or more multi-omic data indices, wherein the cancer analytics comprises tumor characteristics selected from the group consisting of quality control, tumor mutation burden, genomic mutation signatures, microsatellite instability status, neo-antigens, HLA-allele typing, RNA confirmed variants, copy number variants, structural variants, non-coding regulatory variants, gene fusions, pathway enrichment, cancer driver identification, mutation summary, differential gene expression, immune signatures, matching information about treatment outcomes for similar patients and combinations thereof.

75. The system of claim 74, wherein the cancer analytics are derived for an individual sample or a cohort of samples.

76. The system of claim 74, wherein the cancer analytics comprises machine learning predictions and ranked features.

77. The system of claim 76, wherein the machine learning predictions are selected from the group consisting of a primary site of origin classifier, a prediction of future metastasis site classifier, prediction of microsatellite instability status, prediction of neo-antigen binding affinities, disease state stratification, determining cancer lineages, and combinations thereof.

78. The system of claim 71, wherein the indexing engine is configured to propagate annotations from higher levels of genomic hierarchy to lower levels of genomic hierarchy.

79. The system of claim 71, wherein the ranking engine is configured to rank the selected one or more multi-omic data indices from higher levels of genomic hierarchy to lower levels of genomic hierarchy.

80. The system of claim 71, wherein the rank comprises a clinical rank for cancer variants and genes.

81. The system of claim 71, wherein the rank comprises stratifying a cohort by incorporating a latent space representation for cancer data.

82. The system of claim 81, wherein the cohort is stratified into responders and non-responders.

83. The system of claim 81, wherein the cohort is stratified into long-progression free survival time and short-progression free survival time.

84. The system of claim 79, wherein the cohort is stratified into different cancer sub-types.

85. The system of claim 81, wherein the latent space representation is performed by a neural network.

86. The system of claim 85, wherein the neural network is selected from the group consisting of autoencoders, variational autoencoders, deep belief networks, restricted Boltzman machines, feed forward, convolutional, recurrent, gated recurrent, long short-term memory, residual, and generative adversarial networks.

87. The system of claim 71, wherein the ranking engine further comprises a model for learning to rank selected from the group consisting of support vector machines, boosted decision trees, regression models, neural networks, and combinations thereof.

88. The system of claim 71, wherein the rank further comprises a deep learning rank.

89. The system of claim 88, wherein the deep learning rank is derived from a deep learning model selected from the group consisting of a deep semantic similarity model, a deep and wide model, a deep language model, a learned deep learning text embedding, a learned named entity recognition, Siamese neural network, and combinations thereof.

90. The system of claim 71, wherein the multi-omic data is selected from the group consisting of somatic calls from whole genome sequence data, somatic calls from whole exome sequence data, somatic panel sequencing from fresh frozen tissue, somatic panel sequencing from formalin-fixed paraffin-embedded tissue, somatic panel sequencing from liquid biopsy, tumor and normal variant calls, tumor/normal transcriptomic data indexed as variant confirmed in RNA or gene expression level, epigenetic data, chromatin accessibility data, microbiomic data, proteomic data, single cell sequencing data, and combinations thereof.

91. The system of claim 71, wherein the multi-omic data indices further comprise extracted phenotype data.

92. The system of claim 91, wherein the phenotype data is selected from the group consisting of electronic health records, clinical data, functional data, and combinations thereof.

93. The system of claim 71, wherein the multi-omic data indices further comprise featurized imaging data.

94. The system of claim 93, wherein the featurized imaging data is selected from the group consisting of histology slides, MRI images, X-rays, mammograms, ultrasounds, PET images, CT scans, and combinations thereof.

95. The system of claim 74, wherein the cancer analytics are dynamically computed after receipt of the user query.

96. The system of claim 71, wherein the indexing engine is further configured to index derived data selected from the group consisting of cancer analytics, annotations, features extracted from imaging data, phenotypic, medical literature data and its embeddings, and combinations thereof.

97. The system of claim 71, wherein the ranking engine is further configured to match sample alterations with established drug target labels and available clinical trials.

98. The system of claim 71, wherein the ranking engine is further configured to identify cancer drug targets in cohorts by detecting a potential biomarker that stratifies the cohort based on a clinical variable of interest and/or statistical significance, and further configured to the return the ranked one or more multi-omic data indices to the user via a stratification visualization.

99. The system of claim 71, wherein the ranking engine is configured to return the ranked one or more multi-omic data indices to the user via a dynamic creation of hyper-linked reports for individual patients and/or cohorts that provide comprehensive profiling of a tumor.

100. The system of claim 71, wherein the user query comprises user-uploaded data selected from the group consisting of a panel of variants, genes, pathways, disease state conditions, phenotypes of interest, and wherein the selecting comprises querying individual sample or cohort data sub-selected by the uploaded data.

101. The system of claim 71, wherein the user interface is configured to receive a user query that comprises uploaded data for indexing, the data selected from the group consisting of genomic data, transcriptomic data, epigenetic data, chromatin accessibility data, microbiomic data, proteomic data, phenotypic data, annotation data, and combinations thereof.

102. The system of claim 71, wherein the query engine is further configured to normalize and/or expand the user query, classify the intent of the query, summarize retrieved documents, and perform document retrieval based on the similarity between the query and a document in a latent space using deep learning methods.

103. The system of claim 71, wherein at least one of the indexing engine, query engine and ranking engine is configured to utilize deep neural networks.

104. The system of claim 74, wherein cancer analytics engine is configured to derive the cancer analytics utilizing deep neural networks.

105. The system of claim 71, wherein the ranking engine is further configured to return the ranked one or more multi-omic data indices to the user further by returning a summary visualization of the returned results along with the list of ranked results.

106. A system for utilizing multi-omic data indices for tumor profiling, the system comprising
an indexing unit comprising:
a storage element configured to store a plurality of multi-omic data indices, wherein each of the plurality of multi-omic data indices comprises cancer-specific tokenized data, and
an indexing engine configured to
ingest additional multi-omic data and any annotation associated with the additional multi-omic data, the additional multi-omic data related to one or more indices, and
index the ingested additional multi-omic data and annotation while preserving gene names, gene variant names and multi-omic mapping between different data streams for the same patient in the specific index, to produce tokenized ingested additional multi-omic data;
a user interface configured to receive a user query; and
a query engine configured to select one or more relevant multi-omic data indices from the indexing unit based on the user query, rank the selected one or more multi-omic data indices based on at least one of clinical actionability, pathogenicity, feature weight, or frequency, and return the ranked one or more multi-omic data indices to the user via the user interface.
